(19) United States
(12) Patent Application Publication
(10) Pub. No.: US 2018/0116541 A1
(43) Pub. Date: May 3, 2018

(54) System and Method for Monitoring and Treating Arrhythmia and Cardiac Function via the Intrinsic Cardiac Nervous System

(71) Applicant: THE REGENTS OF THE UNIVERSITY OF CALIFORNIA, Oakland, CA (US)

(72) Inventors: Kalyanam Shivkumar, Los Angeles, CA (US); Jeffrey L. Ardell, Los Angeles, CA (US)

(21) Appl. No.: 15/568,073

(22) PCT Filed: Apr. 21, 2016
(86) PCT No.: PCT/US16/28591

§ 371 Date: Oct. 20, 2017

Related U.S. Application Data
(60) Provisional application No. 62/150,463, filed on Apr. 21, 2015.

(51) Int. Cl. A61B 5/0464; A61B 5/04; A61N 1/36; A61B 5/00

(52) U.S. Cl. CPC A61B 5/0464; A61B 5/04001; A61N 1/0551; A61B 5/4836; A61N 1/36114

(57) ABSTRACT

The present invention includes systems and methods for measuring, monitoring and treating arrhythmia and cardiac function via the intrinsic cardiac nervous system. The systems and methods compare neural signatures of the intrinsic cardiac nervous system that are associated with both healthy and diseased myocardial tissue to identify and target afferent intrinsic cardiac neurons for which signaling has been affected by diseased cardiac tissue. Accordingly, methods of modulating afferent neural signals from the diseased myocardium to the intrinsic cardiac nervous system, intrathoracic extracardiac ganglia, and higher centers of the cardiac neuraxis are used as a novel therapeutic approach to mitigating ischemic heart disease.

### CROSS-REFERENCES TO RELATED APPLICATIONS

This application claims priority to U.S. Provisional Patent Application No. 62/150,463, filed on Apr. 21, 2015, the contents of which are incorporated by reference herein in its entirety.

### BACKGROUND OF THE INVENTION

Sudden cardiac death (SCD) due to ventricular arrhythmias is the leading cause of mortality in the world, resulting in an estimated four to five million deaths each year (Chugh et al., 2008, Progress in cardiovascular diseases, 51(3):213-28). Dysregulation of the autonomic nervous system (ANS) following myocardial infarction (MI) plays a crucial role in the genesis of arrhythmias and progression to heart failure (Vaseghi and Shivkumar, 2008, Prog Cardiovasc Dis, 50(6):404-19; Shen and Zipes, 2014, Circulation Research; 114(6):1004-21). The cardiac neuraxis is responsible for the dynamic regulation of cardiac electrical and mechanical function (Armour, 2004, Am J Physiol Regul Integr Comp Physiol; 287(2):R262-71; Ardell, 2004, Basic and Clinical Neurocardiology. New York: Oxford University Press: 187-219), and involves neural networks located from the level of the heart (Armour, 2008, Exp Physiol; 93(2):165-76; Beaumont et al., 2013, The Journal of physiology; 591(Pt 18):4515-33) to that of the insular cortex (Oppenheimer and Hopkins, 1994, Neurocardiology. New York: Oxford University Press: 309-42; Gray et al., 2007, Proc Natl Acad Sci USA., 104(16):6818-23).

At the organ level, the intrinsic cardiac nervous system (ICNS) comprises a distributed network of ganglia and interconnecting nerves (Armour, 2008, Exp Physiol; 93(2):165-76). The ICNS, in concert with higher neuraxial centers (intrathoracic extracardiac ganglia, spinal cord, brain stem, and cortex), regulates cardiac function on a beat-to-beat basis (Armour, 2004, Am J Physiol Regul Integr Comp Physiol.; 287(2):R262-71; Ardell, 2004, Basic and Clinical Neurocardiology. New York: Oxford University Press; 187-219; Beaumont et al., 2013, The Journal of physiology.; 591(Pt 18):4515-33). The ICNS contains all the neural elements necessary for intracardiac reflex control independent of higher centers (Murphy et al., 2000, Ann Thorac Surg.; 69(6):1769-81), namely sensory neurons, adrenergic and cholinergic efferent postganglionic neurons, as well as interposed local circuit neurons (LCNs) (Armour, 2008, Exp Physiol.; 93(2):165-76). The largest subpopulation, LCNs, account for the intra- and interganglionic communication that occurs among neurons within the ICNS and is responsible for local information processing (Armour, 2008, Exp Physiol., 93(2):165-76).

Cardiac disease, such as MI, adversely affects the myocardium and impacts its associated neural components (Vracko et al., 1991, Hum Pathol.; 22(2):138-46; Cao et al., 2000, Circulation. 101(16):1960-9; Ajijola et al., 2015, Heart Rhythm.; Kember et al., 2013, Physiol Genomics; 45(15):638-44). Neural signals regarding cardiac injury are transduced by cardiac afferents to multiple levels of the cardiac neuraxis (Armour, 1999, Cardiovascular Research.; 41(1):41-54). Neural remodeling within the cardiac neuraxis, and its processing of that sensory signal post-MI (Wang et al., 2014, Hypertension.; 64(4):745-55), contributes to neurohumoral activation (Zucker et al., 2012, Heart failure clinics.; 8(1):87-99) and the potential for SCD. Intrinsic cardiac (IC) neurons from humans with ischemic heart disease contain inclusions and vacuoles, as well as display degenerative changes in their dendrites and axons (Hopkins et al., 2000, The Anatomical record.; 259(4):424-36). In vitro intracellular studies of IC neurons derived from chronic MI animals show enhanced excitability, altered synaptic efficacy, and adaptive changes in neurochemical phenotypes (Hardwick et al., 2014, Auton Neurosci; 181(4-12). However, the functional consequences of such changes on neural signaling in vivo, in the context of a healed infarct have not been directly recorded and there is limited knowledge.

What is needed is a thorough examination of morphological and phenotypic remodeling within the ICNS, and evaluations of remodeling of ICNS processing of afferent and efferent inputs (sympathetic and parasympathetic), and their integration by LCNs. The effects of cardiac pacing also needs further review. Pacing has direct relevance to understanding from a mechanistic perspective the reason why strategies to minimize ventricular pacing in patients with devices have been shown in clinical trials to impart survival benefit in patients with ischemic heart disease (Wilkoff et al, 2002, JAMA.; 288(24):3115-23; Sweeney et al., 2003, Circulation.; 107(23):2932-7).

There is a need for systems and methods for characterizing adverse neural signatures associated with ischemic heart disease to aid in monitoring disease and the efficacy of therapies to target the ANS and mitigate the progression of cardiac disease. The present invention satisfies this need.

### SUMMARY OF THE INVENTION

In one aspect, the present invention provides a method of assessing ischemic heart disease in a subject. The method comprises measuring a plurality of electrical signals indicative of a neural signature from at least one intrinsic cardiac neuron; comparing the measured signals to a reference neural signature of the intrinsic cardiac nervous system; and determining if the difference between the measured signals and the neural signature exceeds a threshold value.

In one embodiment, the method comprises treating the subject with at least one therapeutic element when the threshold value is exceeded. In one embodiment, the at least one therapeutic element is a drug or biological agent. In one embodiment, the at least one therapeutic element is an electrical stimulus to a region of the subject's myocardial tissue or to one or more intrinsic cardiac neurons.

In one embodiment, the method comprises detecting the relative amount of afferent neurons in a population of intrinsic cardiac neurons.

In one embodiment, the reference neural signature is specific to the subject. In one embodiment, the reference neural signature is based on a subject population having at least one common characteristic selected from the group consisting of gender, age, activity level, diet, congenital defect, genetic trait, and metabolic status.

In one embodiment, the measured neural signature includes at least one parameter selected from the group consisting of intrinsic neuron spontaneous firing rate, activity during cardiac cycle phases, temporal relationship between neurons, response to mechanosensitive input, response to stimulation of the sympathetic or parasympathetic nervous system, change in cardiac loading conditions, response to epicardial pacing, response to chemoreceptor stimulation, and response to noceptive input.

In one aspect, the present invention provides a method of treating a subject having diseased myocardium. The method comprises identifying diseased myocardial tissue in the subject's heart; identifying at least one afferent intrinsic cardiac neuron signaling from the diseased myocardial tissue; and modifying the signaling from the identified afferent intrinsic cardiac neuron.

In one embodiment, identifying at least one afferent intrinsic cardiac neuron signaling from the diseased myocardial tissue comprises comparing electrical signals measured from afferent neurons to a neural signature of the intrinsic cardiac nervous system.

In one embodiment, modifying the signaling from the identified afferent intrinsic cardiac neuron comprises applying an electrical stimulus to the afferent intrinsic cardiac neuron.

In one embodiment, the method is used in a closed loop system for monitoring and treating ischemic heart disease in the subject.

### BRIEF DESCRIPTION OF THE DRAWINGS

The following detailed description of preferred embodiments of the invention will be better understood when read in conjunction with the appended drawings. For the purpose of illustrating the invention, there are shown in the drawings exemplary embodiments and experimental data supportive of the studies performed herein. It should be understood, however, that the invention is not limited to the precise arrangements and instrumentalities of the embodiments shown in the drawings.

FIG. 1 is a flowchart of an exemplary method of the present invention.

FIG. 2 is a flowchart of another exemplary method of the present invention.

FIG. 3, comprising FIG. 3A through FIG. 3K, is a set of images demonstrating myocardial infarction induces morphological and phenotypic remodeling of intrinsic cardiac neurons. FIG. 3A are photomicrographs showing hematoxylin and eosin (H&E) stained neurons from the ventral interventricular ganglionated plexus (VIV GP) in control versus myocardial infarction (MI) animals. FIG. 3B is a histogram of intrinsic cardiac neural size distribution in control versus MI animals. FIG. 3C depicts mean IC neural size in control versus MI animals. FIG. 3D are photomicrographs showing VIV GP stained with choline acetyltransferase (ChAT) in control versus MI animals. ChAT catalyzes the synthesis of acetylcholine and was used to identify putative cholinergic neurons. FIG. 3E depicts percentage of ChAT-positive IC neurons in control versus MI animals. FIG. 3F are photomicrographs showing VIV GP stained with tyrosine hydroxylase (TH) in control versus MI animals. TH catalyzes the rate-limiting step in the synthesis of norepinephrine and was used to identify putative adrenergic neurons (black arrowheads). FIG. 3G shows the percentage of TH-positive IC neurons in control versus MI animals. FIG. 3H are photomicrographs showing VIV GP stained with vasoactive intestinal peptide (VIP) in control versus MI animals. VIP is an important modulator of cardiac function and a marker of putative afferent neurons. FIG. 3I shows the percentage of VIP-positive neurons in control and MI animals. FIG. 3J is an image of porcine heart with chronic anteroapical MI. The location of the VIV GP in relation to the infarct scar (white dashed scar) is shown. FIG. 3K shows the corresponding short axis cardiac magnetic resonance image of heart. White arrow indicates areas of delayed hyperenhancement resulting from scar tissue. Scale bar in all photomicrographs=50 μm.

FIG. 4, comprising FIG. 4A through FIG. 4E, is a set of images showing that myocardial infarct induces differential morphologic and neurochemical remodeling of intrinsic cardiac neurons. FIG. 4A is a schematic showing location of right atrial ganglionated plexus (RA GP), right marginal artery ganglionated plexus (RMA GP), dorsal interventricular ganglionated plexus (DIV GP), and VIV GP. The RA GP and RMA GP predominantly exert influence over the right atrium and RV, respectively, whereas the DIV GP and VIV GP predominantly exert influence over the LV. FIG. 4B are photomicrographs showing H&E stained neurons from the ganglionated plexi studied in control versus MI animals. FIG. 4C shows the mean IC neural size in the ganglionated plexi in control versus MI animals. FIG. 4D are photomicrographs showing the ganglionated plexi stained with VIP in control versus MI animals. FIG. 4E shows the percentage of VIP-positive neurons in the ganglionated plexi in control versus MI animals. Scale bar in all photomicrographs=50 μm.

FIG. 5, comprising FIG. 5A through FIG. 5D, is set of images relating to the experimental methods used for intrinsic cardiac neural recording. FIG. 5A is a schematic showing location of VIV GP, from which neural activity was recorded. FIG. 5B is a schematic of a 16-channel linear microelectrode array (LMA) used to record in vivo activity of multiple individual neurons contained within the VIV GP. FIG. 5C is a representative trace showing the activity of two IC neurons (ICNs) identified from a single channel of the LMA. FIG. 5D shows basal activity of the IC neurons from panel 5A in relation to the cardiac cycle. Note that the activity of both neurons is clustered predominantly during systole. ECG=electrocardiogram; LVP=left ventricular pressure.

FIG. 6, comprising FIG. 6A through FIG. 6C, is a set of images regarding the analytics and functional classification of intrinsic cardiac neurons. FIG. 6A shows spiking activity recorded from 5 IC neurons in a control heart. Vertical dotted lines indicate the onset and offset of left ventricular (LV) epicardial mechanical stimulus. Note that subpopulations of neurons showed an increase (ICN 2), a decrease (ICN 3, 4, and 5), or no change in activity (ICN 1) from baseline. The significance levels of induced changes in activity are shown to the right of each trace. P values were derived based on the analysis described in the Methods. FIG. 6B is a summary of evoked changes in IC neural activity in response to cardiovascular stimuli in a MI animal. Horizontal rows represent the response of an individual neuron to a given stimulus (vertical columns). Green indicates significant increases in activity (p<0.05); red indicates significant decreases (p<0.05). FIG. 6C is a functional classification of IC neurons depicted in FIG. 6B. Neurons were classified as afferent, efferent, or convergent based on their responses to the cardiovascular stimuli. Afferent neurons were defined as those that responded solely to: epicardial mechanical stimuli of the right (RV) or left ventricle (LV); transient occlusion of the inferior vena cava (IVC); and/or transient occlusion of descending thoracic aorta. Efferent neurons were defined as those that responded solely to: electrical stimulation of the left (LCV) or right cervical vagus (RCV); electrical stimulation of the left (LSG) or right stellate ganglia (RSG); and/or transient occlusion of the bilateral carotid arteries (BCA). Neurons that responded to activation of both afferent and efferent inputs were defined as convergent.

FIG. 7, comprising FIG. 7A through FIG. 7C, is a set of images demonstrating that myocardial infarction induces no overall change in functional or temporal characteristics of intrinsic cardiac neurons. FIG. 7A is a histogram of baseline firing rates of IC neurons identified in control versus MI hearts. FIG. 7B is a functional classification of IC neurons in control versus MI hearts. FIG. 7C shows cardiac cycle-related periodicity of IC neurons in control versus MI hearts. Note that subpopulations of neurons displayed diastolic-related activity, systolic-related activity, diastolic- and systolic-related activity, or stochastic behavior. MI did not significantly alter the functional or temporal characteristics of the neurons. Chi-square test was used to determine significance between groups.

FIG. 8, comprising FIG. 8A through FIG. 8F, is a set of images demonstrating that myocardial infarction induces afferent and efferent remodeling of intrinsic cardiac neurons. FIG. 8A shows that epicardial mechanical stimuli was used to assess the capacity of IC neurons to transduce mechanosensitive afferent inputs arising from the RV or LV. FIG. 8B shows the percentage of IC neurons receiving mechanosensitive inputs arising from the LV apex (infarct zone) versus RV and LV (border and remote zones) in control versus MI hearts. FIG. 8C shows the percentage of IC neurons responding to transient IVC or aortic occlusion in control versus MI hearts. IVC and aortic occlusions were used to assess the capacity of neurons to transduce changes in preload and afterload, respectively. FIG. 8D shows the percentage of IC neurons transducing multiple afferent inputs in control versus MI hearts. FIG. 8E shows the percentage of IC neurons receiving efferent inputs from parasympathetic and/or sympathetic preganglionic neurons, as assessed by cervical vagus nerve stimulation (VNS) and stellate ganglia stimulation (SGS), respectively, in control versus MI hearts. FIG. 8F shows the percentage of IC neurons responding to transient bilateral carotid artery occlusion in control versus MI hearts. Bilateral carotid artery occlusion was used to assess the capacity of the baroreflex to modulate efferent inputs to neurons. Chi-square test was used to determine significance between groups.

FIG. 9, comprising FIG. 9A through FIG. 9C, is a set of images demonstrating the analytics and functional classification of IC neurons responsive to pacing. FIG. 9A shows spiking activity recorded from 5 IC neurons in a control animal. Vertical dotted lines indicate the onset and offset of epicardial pacing at the right ventricular outflow tract (RVOT). Note that subpopulations of neurons showed an increase, a decrease, or no change in activity from baseline. The significance levels of induced changes in activity for each neuron are shown to the right of the trace. FIG. 9B is a summary of evoked changes in IC neural activity in response to regional epicardial pacing in a MI animal, along with responses to other cardiovascular stimuli. Green indicates significant increases in activity (p<0.05); red indicates significant decreases (p<0.05). FIG. 9C shows the functional classification of pace-responsive IC neurons depicted in FIG. 9B using the protocol outlined in FIG. 6.

FIG. 10, comprising FIG. 10A and FIG. 10B, is a set of images demonstrating that myocardial infarction alters response and characteristics of intrinsic cardiac neurons to pacing. FIG. 10A shows the percentage of IC neurons responding to epicardial pacing at the right atrial appendage (RAA), RVOT, RV apex, or LV apex in control versus MI hearts. MI induced a differential decrease in the neural response to ventricular versus atrial pacing. FIG. 10B shows a functional classification of pace-responsive IC neurons in control versus MI animals. MI altered the response characteristics to pacing at both infarct (LV apex) and remote zones (RVOT). Chi-square test was used to determine significance between groups.

FIG. 11 is a set of images demonstrating the state dependence of intrinsic cardiac neurons. IC neural activity at baseline (BL) and in response to epicardial pacing at the RAA, RVOT, RV apex, or LV apex in control versus MI animals. Neurons are subdivided based on evoked increases versus decreases in activity in response to pacing. Neurons that had a low basal activity were activated by pacing, while those with a high basal activity were suppressed, suggesting a state-dependent nature. *=P<0.01.

FIG. 12 is a table showing the firing characteristics of IC neurons to evoked stressors. IC neural activity (mean±SE) at baseline and in response to the cardiovascular stimuli in control versus MI animals. Neurons are subdivided based on evoked increases (upper panel) versus decreases (lower panel) in activity in response to a given stimuli.

FIG. 13, comprising FIG. 13A and FIG. 13B, is a set of images demonstrating a myocardial infarct model. FIG. 13A are angiographic images before (left panel) and after (right panel) occlusion of left anterior descending coronary artery at the level of the third diagonal branch (white arrowhead) using microembolization technique. FIG. 13B is a lead II of an electrocardiogram before (upper panel) and acutely after (lower panel) the infarction showing ST-segment elevation.

FIG. 14, comprising FIG. 14A through FIG. 14D, depicts an analysis demonstrating that myocardial infarction reduces functional network connectivity within the intrinsic cardiac nervous system. FIG. 14A depicts the conditional probability that an IC neuron that responded to one stimulus (X, x-axis) also responded to another stimulus (Y, y-axis) in control animals. FIG. 14B is a graphical representation of interdependent interactions between stimuli in control animals. FIG. 14C depicts the conditional probability that a neuron that responded to one stimulus (X, x-axis) also responded to another stimulus (Y, y-axis) in MI animals. FIG. 14D is a graphical representation of interdependent interactions between stimuli in MI animals. Color scale in FIG. 14A and FIG. 14C indicates level of probability of each occurrence. Arrow thickness in FIG. 14B and FIG. 14D proportional to the strength of conditional probability. Only links with probabilities≥0.6 are displayed. Afferent and efferent stimuli are represented by blue and red, respectively. Atrial (A pace) and ventricular pacing (V pace) are represented by black.

FIG. 15, comprising FIG. 15A and FIG. 15B is as schematic representation of the functional remodeling of the intrinsic cardiac nervous system post-myocardial infarction. FIG. 15A is a schematic diagram showing neural connections between the intrinsic cardiac nervous system (ICNS) and the heart, as well as inputs from higher centers of the cardiac neuraxis, in health. FIG. 15B is schematic diagram showing the alterations in neural connections between ICNS and heart that occur following MI. There is an increase in sympathetic and parasympathetic inputs to convergent local circuit neurons (LCNs), while there is a decrease in afferent inputs from the infarct compared with border and remote regions of the heart. Green and red dashed and continuous lines represent pre- and postganglionic fibers, respectively.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical systems and methods for monitoring and treating arrhythmia and cardiac function. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

Contemplated herein are systems and methods for measuring, monitoring and treating arrhythmia and cardiac function via the intrinsic cardiac nervous system. In certain embodiments, the systems and methods compare neuronal signatures of the intrinsic cardiac nervous system that are associated with both healthy and diseased myocardial tissue to identify and target intrinsic cardiac neurons for which signaling has been affected by diseased cardiac tissue. Accordingly, modulation of afferent neural signals from the diseased myocardium to the intrinsic cardiac nervous system, intrathoracic extracardiac ganglia, and higher centers of the cardiac neuraxis represents a novel therapeutic approach to mitigating ischemic heart disease. Modulation of processing of efferent input within the ICNS represents a novel therapeutic approach to mitigate ischemic heart disease. This modulation can take place in the direct pathways—preganglionic to postganglionic neurons or via the pathways that involve intra-ganglionic and inter-ganglionic neural interactions.

In one exemplary embodiment, the present invention includes a system for measuring, modulating, and/or stimulating intrinsic cardiac neural signaling. The system may include at least one implantable or partially implantable sensor incorporating a plurality of electrodes for detecting electrical signals generated by intrinsic cardiac neurons For example, in one embodiment, the sensor comprises a linear microelectrode array (LMA). In certain embodiments, the LMA comprises a plurality of electrodes. For example, in one embodiment, the LMA comprises 16 platinum/iridium electrodes. The sensor may comprise any suitable type and size of electrode suitable for detecting electrical signals in one or more intrinsic cardiac neurons. Exemplary electrodes include, but are not limited to, single shank electrodes, 2D multi-shank electrodes, 3D multi-shank electrodes, and multielectrode arrays.

The same or different electrodes may be used for applying focal electrical stimulus to any intrinsic cardiac neuron, or to any region of myocardial tissue. These electrodes may be designed for insertion into (or to make contact with) the intrinsic cardiac neurons or ganglia of a subject to effectively detect electrical activity of the neurons for recording at a sensor control unit connected to the electrodes. While the electrodes are implantable in a subject, the control unit may either be implantable in the subject or external to the subject, as desired.

In one embodiment, the sensor may comprise one or more pre-amplifiers, amplifiers, or filters to process the detected electrical signal. Such components may be positioned on an implanted sensor, or alternatively be present on external hardware. For example, in one embodiment, the preamplifier provides for low and high pass filtering with gain control. In one embodiment, the filtering range is 300 to 3 KHz with gain up to 5K. In certain embodiments, the filtering range and/or gain of the preamplifier is adjustable to optimize signal to noise ratio. In one embodiment, the preamplifier and control device allow for transient blocking of input signal as related to electrical stimuli or electrical activity generated by atrial or ventricular tissues.

Accordingly, the sensor control unit may be powered by any method understood in the art, including a standard battery, standard wiring for external power transfer, or it may include a receiver coil for wireless power transfer. The control unit may include a microprocessor and any form of memory for storing control software and any received and/or processed data. The control unit may further include a transceiver or any hardware and software necessary for transmitting and/or receiving data with an external processing unit for further analysis of the recorded activity within each neuron being measured. The external processing unit may be one or more computing units, and may be or include any type of computing device including a desktop laptop, tablet, smartphone or other wireless digital/cellular phones, wrist watches, televisions or other thin client device as would be understood by those skilled in the art. Generally, any computing devices described herein may include at least one processor, standard input and output devices, as well as all hardware and software typically found on computing devices for storing data and running programs, and for sending and receiving data over a network, if needed. It should also be appreciated that the recorded data may be further filtered (such as, amplified or any other type of additional processing for analyzing and displaying the data as desired by the external processing unit or other connected computing device within the system.

In one example and without limitation, the system may utilize a linear microelectrode array (LMA), such as that produced by MicroProbes in Gaithersburg, Md. Briefly, the LMA may include 16 platinum/iridium electrodes having a diameter of about 25 μm with an exposed tip of about 2 mm, and impedance of 0.3-0.5 MΩ at 1 kHz. However, it should be appreciated that the systems of the present invention may use any sensor/electrode set understood by those skilled in the art, provided such electrode units are suitable for connecting to intrinsic cardiac neurons. These include, but are not limited to, single shank, 2D and 3D shank electrodes as well as planer electrodes overlaying intrinsic cardiac ganglia and their projections to atrial and ventricular tissues.

In certain embodiments, the system of the invention comprises one or more components to stimulate the afferent intrinsic cardiac neurons. For example, in one embodiment, the system comprises a component suitable for delivery of a mechanical force to the myocardial tissue, including but not limited to a blunt object (e.g., catheter, electrode, needle, rod, etc.) or a fluid (liquid or gas) deliverable to the tissue. In one embodiment, the one or more components are used to focally inject chemicals to alter activity on afferent neurons. In one embodiment, the system comprises one or more components to alter the preload or afterload. In certain embodiments, the system comprises one or more stimulatory electrodes to apply an electrical signal to the sympathetic or parasympathetic nervous system, used to stimulate the efferent intrinsic cardiac neurons. Exemplary electrodes include cuff electrodes, needle electrodes, and the like. In one embodiment, the system comprises one or more pacing electrodes suitable for application of cardiac electrical stimulation at one or more epicardial sites.

The system may further include a software platform with a graphical user interface (GUI) for modulating the function of the one or more sensors and for displaying information regarding the historical or real-time electrical activity of the measured intrinsic cardiac neurons, as well as historical or real-time measurement of the subject's cardiac function. In certain embodiments, the wireless communication information transfer to and from the sensor control unit and the external processing unit may be via a wide area network and may form part of any suitable networked system understood by those having ordinary skill in the art for communication of data to additional computing devices, such as, for example, an open, wide area network (e.g., the internet), an electronic network, an optical network, a wireless network, a physically secure network or virtual private network, and any combinations thereof. Such an expanded network may also include any intermediate nodes, such as gateways, routers, bridges, internet service provider networks, public-switched telephone networks, proxy servers, firewalls, and the like, such that the network may be suitable for the transmission of information items and other data throughout the system.

As would be understood by those skilled in the art, the external processing unit may be wirelessly connected to the expanded network through, for example, a wireless modem, wireless router, wireless bridge, and the like. Additionally, the software platform of the system may utilize any conventional operating platform or combination of platforms (Windows, Mac OS, Unix, Linux, Android, etc.) and may utilize any conventional networking and communications software as would be understood by those skilled in the art.

To protect data, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access. Access or use restrictions may be implemented for users at any level. Such restrictions may include, for example, the assignment of user names and passwords that allow the use of the present invention, or the selection of one or more data types that the subservient user is allowed to view or manipulate.

In certain embodiments the network provides for telemetric data transfer from the sensor control unit to the external processing unit, and vice versa. For example, data transfer can be made via any wireless communication and may include any wireless based technology, including, but not limited to radio signals, near field communication systems, hypersonic signal, infrared systems, cellular signals, GSM, and the like. In some embodiments, data transfer is conducted without the use of a specific network. Rather, in certain embodiments, data is directly transferred to and from the sensor control unit and external processing unit via systems described above.

The software may include a software framework or architecture that optimizes ease of use of at least one existing software platform, and that may also extend the capabilities of at least one existing software platform. The software provides applications accessible to one or more users (e.g. patient, clinician, etc.) to perform one or more functions. Such applications may be available at the same location as the user, or at a location remote from the user. Each application may provide a graphical user interface (GUI) for ease of interaction by the user with information resident in the system. Exemplary GUIs of the invention may include the ability for a user to control the function or mode of the sensors, as well as the ability to display individual intrinsic cardiac neuron activity, pooled data of neuronal activity, or of general cardiac function as would be understood by those skilled in the art. Such data may include indices of network function including, but not limited to, temporal relationships of neural activity to one another, temporal relationships to cardiac electrical or mechanical events, temporal relationships to controlled events including pacing, mechanical, or chemical stressors. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system. Presentation of data through the software may be in any sort and number of selectable formats. For example, a multi-layer format may be used, wherein additional information is available by viewing successively lower layers of presented information. Such layers may be made available by the use of drop down menus, tabbed folder files, or other layering techniques understood by those skilled in the art.

The software may also include standard reporting mechanisms, such as generating a printable results report, or an electronic results report that can be transmitted to any communicatively connected computing device, such as a generated email message, text or file attachment. Likewise, particular results of the aforementioned system can trigger an alert signal, such as the generation of an alert email, text or phone call, to alert a patient, doctor, nurse, emergency medical technicians, or other health care provider of the particular results.

Using the systems described herein, the present invention further includes methods for determining intrinsic cardiac neuron signal activity within the intrinsic cardiac nervous system, as well as methods of treating arrhythmia and cardiac function in a subject.

As demonstrated herein, intrinsic cardiac neurons of the intrinsic cardiac nervous system can be individually and/or collectively monitored for signal activity to arrive at a neural signature indicative of healthy myocardial tissue, diseased myocardial tissue, or the neural signature may be indicative of a compromised state in which the subject is at risk of arrhythmia, myocardial ischemia or the general degrading of cardiac function.

In one exemplary embodiment, the neural signature may serve as a baseline or reference signature, meaning that the neural signature represents a profile of a healthy state for use in comparison to subsequent neural signatures taken or measured when the subject is being examined. Baseline or reference neural signatures may be patient specific, or they may be collective or pooled data representative of average values for subjects having at least one characteristic in common. Exemplary characteristics may include patient gender, age, activity level, diet, congenital defect, genetic trait, metabolic status, and the like. In certain embodiments, the baseline or reference neural signature is defined with respect to one or more cardiovascular stressors, including, but not limited to, exercise, orthostatic stress, temperature, Valsalva maneuver, and spirometry test. After establishing a baseline or reference neural signature representative of a healthy state, subsequent measurements of intrinsic cardiac neural activity are taken to establish a real-time neural signature for comparison to the baseline or reference, such that a determination can be made as to whether the subject is in need of a treatment.

As contemplated herein, the neural signature may include one or more parameters, including without limitation, parameters relating to intrinsic neuron spontaneous firing rate, activity during cardiac cycle phases, temporal relationships between neurons, response to mechanosensitive input, change in cardiac loading conditions, response to epicardial pacing, chemoreceptor and noceptive input. For each parameter, a threshold value may be established that is indicative of a subject in need of a treatment, or of a particular type of treatment. In certain embodiments, exceeding only one threshold value may be determinative of a need for treatment and/or type of treatment, whereas in other embodiments, multiple threshold values may be exceeded in order to be determinative of a need for treatment, or particular type of treatment. In still other embodiments, a scoring algorithm may be used to determine whether the differences in neural signature comparisons is demonstrative of a need for treatment, or of a particular type of treatment. In certain embodiments, scoring includes changes in individual or grouped activity, directionality of changes in such activity and temporal relationships between 2 or more neurons

The method may be used to diagnose a cardiac condition, assess the recovery of a cardiac condition, assess the efficacy of a therapy of a cardiac condition, determine the likelihood of a future cardiac event, or determine that a prior cardiac event has occurred.

Exemplary cardiac conditions or events detected or monitored by way of the presently described method includes, but is not limited to ischemic heart disease, myocardial infarction, premature ventricular contraction, arrhythmia, reduced ejection heart failure, preserved ejection heart failure, and the like.

The neuronal signature of the intrinsic cardiac nervous system may be assessed by measuring the one or more parameters in one or more intrinsic cardiac neurons. The one or more intrinsic cardiac neurons assessed by way of the method may be of the ventral interventricular ganglionated plexus (VIV GP), dorsal interventricular glanglionated plexus (DIV GP), right marginal artery ganglionated plexus, right atrial ganglionated plexus, or any other neuronal structure of the intrinsic cardiac nervous system.

In certain embodiments, the method comprises determining the number or percentage of intrinsic cardiac neurons that are afferent, efferent, or convergent neurons. For example, it is demonstrated herein that myocardial infarction induces remodeling of the intrinsic cardiac nervous system which significantly reduced the relative amount of afferent inputs from the infarct region while maintaining afferent inputs from adjacent or remote cardiac regions. This boundary condition represents on embodiment of the neural signature of cardiac disease.

In certain embodiments, the number or percentage of afferent neurons can be assessed by determining which neurons transduce a response to mechanical stimuli of myocardial tissue, change in preload (i.e., by transient IVC occlusion), or change in afterload (i.e., by transient occlusion of the descending aorta). In certain embodiments, the method comprises identifying which neurons transduce a response to mechanical stimuli at various locations, for example stimuli in the infarct region, border zone, and remote regions. Mechanical stimuli may be generated by a applying a force to the myocardial tissue, which may be generated by a blunt object (i.e., electrode, needle, or catheter) or by flow of a liquid or gas on to the tissue. In certain embodiments, the numbers or percentage of afferent neurons can be assessed by determining which neurons transduce a chemical stimuli delivered in proximity to the sensory field of the recorded neuron(s). For example, suitable chemicals may be delivered by catheter or needle to focal areas of myocardial tissue or intrinsic cardiac ganglia.

In certain embodiments, the number or percentage of efferent neurons can be assessed by determining which neurons transduce an electrical stimuli delivered to upstream parasympathetic or sympathetic inputs, including but not limited to stimuli to the vagus, stellate ganglia, or mediastinal ganglia. The delivered stimuli may be of any intensity, frequency, or duration, known to be transduced by typical efferent intrinsic cardiac neurons.

In one embodiment, activity of intrinsic cardiac neurons can increase at rest or in response to cardiovascular stressors when associated with myocardial infarction. In one embodiment, activity of intrinsic cardiac neurons can decrease at rest or in response to cardiovascular stressors when associated with myocardial infarction. In another embodiment, activity can increase in a subset of intrinsic cardiac neurons, can decrease in a subset of intrinsic cardiac neurons, and remain unaltered in a subset of intrinsic cardiac neurons. These changes are reflective of the types of neurons being recorded from (afferent, efferent or convergent neurons), the characteristics of the stressor imposed (e.g. mechanical, chemical, nociceptive), and the structure/function of the nerve/myocyte remodeling in heart disease.

In one embodiment, the temporal relationship of intrinsic cardiac neurons to the cardiac cycle can change with myocardial infarction. This can include those neurons who activity is temporally related to diastole (cardiac relaxation), systole (ejection phase) and isovolumetric contraction and relaxation.

In one embodiment, the temporal relationship of one intrinsic cardiac neuron to another can change with myocardial infarction. This temporal relationship may include intrinsic cardiac neurons on one functional class (e.g. afferent related) or may extend across classes (afferent to efferent, afferent to convergent, efferent to afferent and efferent to convergent).

For example, in one embodiment, the spontaneous firing rate of intrinsic neurons may demonstrate a drop of at least 5% when associated with myocardial diseased tissue. In other embodiments, the spontaneous firing rate of intrinsic neurons may demonstrate varying degrees of changes, for example a drop of at least 10%, at least 15%, at least 20%, at least 25%, at least 30%, and even at least 35% or more, when associated with myocardial diseased tissue. In one embodiment, intrinsic neuron activity during cardiac cycle phases may demonstrate a drop of at least 5% during diastolic-related activity when associated with myocardial diseased tissue. In other embodiments, intrinsic neuron activity during cardiac cycle phases may demonstrate a drop of at least 10%, at least 15% or even at least 20% or more during diastolic-related activity when associated with myocardial diseased tissue. In another embodiment, intrinsic neuron activity during cardiac cycle phases may demonstrate an increase of at least 5% during systolic-related activity when associated with myocardial diseased tissue. In other embodiments, intrinsic neuron activity during cardiac cycle phases may demonstrate an increase of at least 10%, at least 15%, at least 20%, at least 25%, at least 30%, at least 35%, at least 40%, at least 45%, or even at least 50% or more during systolic-related activity when associated with myocardial diseased tissue. In another embodiment, intrinsic neuron activity during cardiac cycle phases may demonstrate a drop of at least 5% in dual diastolic- and systolic-related activity when associated with myocardial diseased tissue. In other embodiments, intrinsic neuron activity during cardiac cycle phases may demonstrate a drop of at least 10%, at least 20%, at least 30%, at least 40%, or even at least 50% or more in dual diastolic- and systolic-related activity when associated with myocardial diseased tissue.

In one embodiment, the response to mechanosensitive input of intrinsic neurons may demonstrate a drop of at least 5% when associated with myocardial diseased tissue. In other embodiments, the response to mechanosensitive input of intrinsic neurons may demonstrate a drop of at least 10%, at least 15%, at least 20%, at least 25%, at least 30%, at least 35%, at least 40%, at least 45%, and even at least 50% or more, when associated with myocardial diseased tissue.

In another embodiment, the ability of intrinsic neurons to transduce changes in cardiac loading conditions may include a drop in neural response to a decrease in preload conditions by at least 5% when associated with myocardial diseased tissue. In other embodiments, the ability of intrinsic neurons to transduce changes in cardiac loading conditions may include a drop in neural response to a decrease in preload conditions by at least 10%, at least 15%, at least 20%, at least 25%, at least 30%, and even at least 35% or more, when associated with myocardial diseased tissue.

In another embodiment, the response of intrinsic neurons to epicardial pacing may include an upregulation of pacing-responsive convergent neurons associated with myocardial diseased tissue. In yet another embodiment, the response of intrinsic neurons to epicardial pacing may include a downregulation of pacing-responsive afferent neurons associated with myocardial diseased tissue.

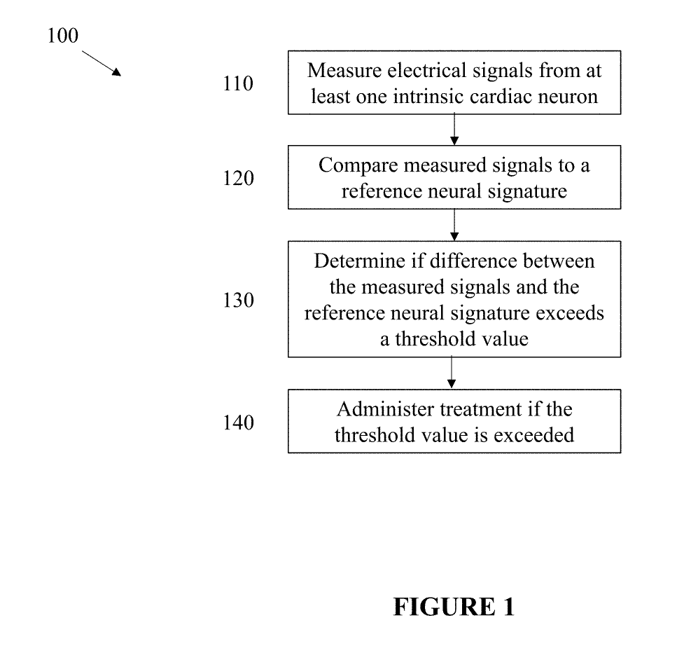

Accordingly, and as shown in FIG. 1, an exemplary method 100 of the invention is illustrated. First, at step 110, a plurality of electrical signals from at least one intrinsic cardiac neuron is measured. Next, at step 120, the measured signals are compared to a reference neural signature of the intrinsic cardiac nervous system. At step 130, it is determined whether the difference between the measured signals and the neural signature exceeds a threshold value. Then, at step 140, if the threshold value is exceeded, the subject is treated with at least one therapeutic element. In certain embodiments, the treatment may be the administration of a drug, compound or other chemical or biological material, while in other embodiments, the treatment may be administration of an electrical stimulus to one or more regions of the heart, including any myocardial tissues or any intrinsic neurons associated therewith. In certain embodiments, the treatment may be administered to extracardiac nexus points including, but not limited to intrathoracic ganglia, the vagosympathetic trunk and spinal cord.

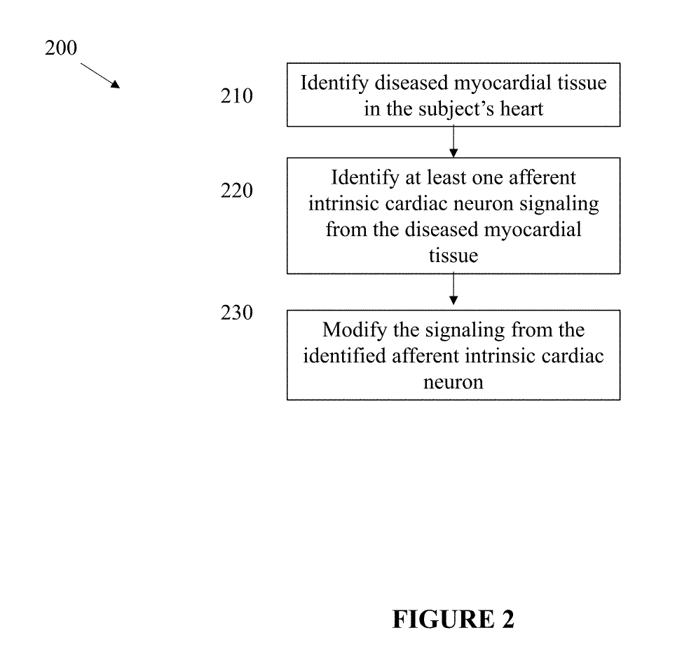

In another exemplary method, as shown in FIG. 2, method 200 may be used for treating a subject previously determined to have a region of diseased myocardium. For example, at step 210, a diseased region of myocardial tissue is identified in the subject's heart. At step 220, at least one afferent intrinsic cardiac neuron signaling from the diseased myocardial tissue is identified, and at step 230, the signaling from the identified afferent intrinsic cardiac neuron is modified. Examples of modifying afferent neurons include, without limitation, application of an electrical stimulus, or administration of a drug, compound or other chemical or biological material. It should be appreciated that the process of identifying at least one afferent intrinsic cardiac neuron signaling from the diseased myocardial tissue may include the process of comparing electrical signals measured from afferent neurons to a neural signature of the intrinsic cardiac nervous system, as described elsewhere herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### Myocardial Infarction Alters Cardiac Neural Signature and Induces Neural Remodeling

Presented herein are experiments designed to examine morphological and phenotypic remodeling within the ICNS and to directly evaluate remodeling of ICNS processing of afferent and efferent inputs (sympathetic and parasympathetic), and their integration by LCNs. The effects of cardiac pacing were also studied. Pacing has direct relevance to understanding from a mechanistic perspective the reason why strategies to minimize ventricular pacing in patients with devices have been shown in clinical trials to impart survival benefit in patients with ischemic heart disease.

It is demonstrated herein that MI induces morphologic and neurochemical changes within the ICNS. This structural remodeling is paralleled by functional alterations in the processing of afferent and efferent neural signals by this neural network. The heterogeneity of afferent neural signals, combined with remodeling of convergent neurons, likely represents the organ level neuropathophysiology of reflex ANS activation that is responsible for arrhythmias and progression to heart failure. Characterization of adverse neural signatures associated with ischemic heart disease can aid in monitoring disease and the efficacy of therapies to target the ANS and mitigate the progression of cardiac disease.

The studies presented herein characterize structural and functional remodeling of the ICNS post-MI in a porcine model (control (n=16) vs. healed anteroapical MI (n=16)). In vivo microelectrode recordings of basal activity, as well as responses to afferent and efferent stimuli, were recorded from intrinsic cardiac neurons. From control 118 neurons and from MI animals 102 neurons were functionally classified as afferent, efferent, or convergent (receiving both afferent and efferent inputs). In control and MI, convergent neurons represented the largest subpopulation (47% and 48%, respectively) and had enhanced transduction capacity following MI. Efferent inputs to neurons were maintained post-MI. Afferent inputs were attenuated from the infarcted region (19% in control vs. 7% in MI; P=0.03), creating a ‘neural sensory border zone’, or heterogeneity in afferent information. MI reduced transduction of changes in preload (54% in control vs. 41% in MI; P=0.05). The overall functional network connectivity, or the ability of neurons to respond to independent pairs of stimuli, within the ICNS was reduced following MI. The neuronal response was differentially decreased to ventricular vs. atrial pacing post-MI (63% in control vs. 44% in MI to ventricular pacing; P<0.01). MI induced morphological and phenotypic changes within the ICNS. The alteration of afferent neural signals, and remodeling of convergent neurons, represents a ‘neural signature’ of ischaemic heart disease.

### Animals

Yorkshire pigs with normal hearts (n=16; 8 male and 8 female; 49±3 kg) and Yorkshire pigs with healed anteroapical MI (n=16; 6 male and 10 female; 46±2 kg) were used in Example 1.

### Creation of Myocardial Infarction

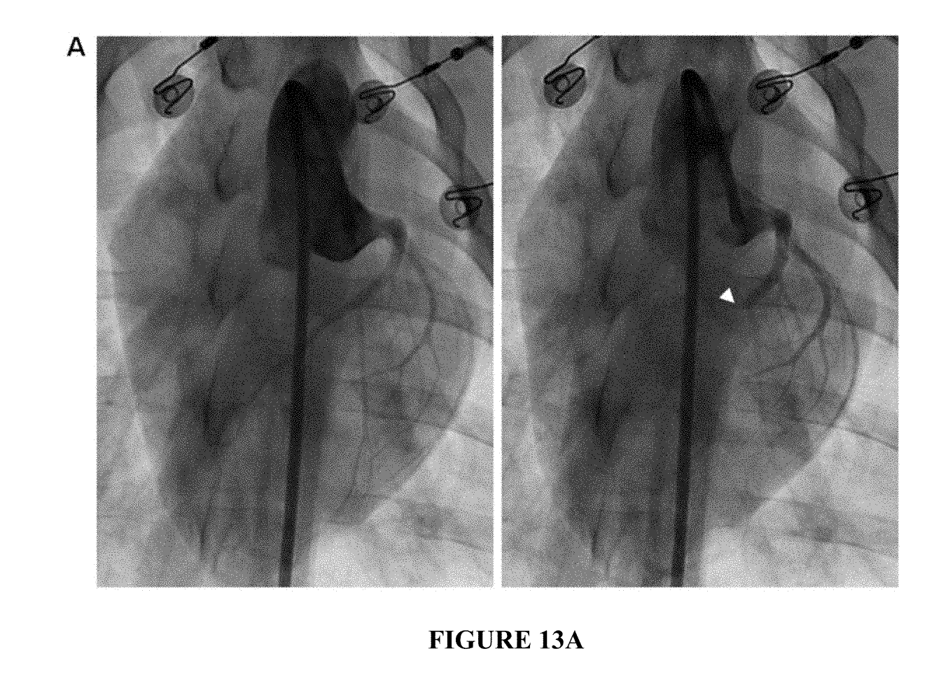

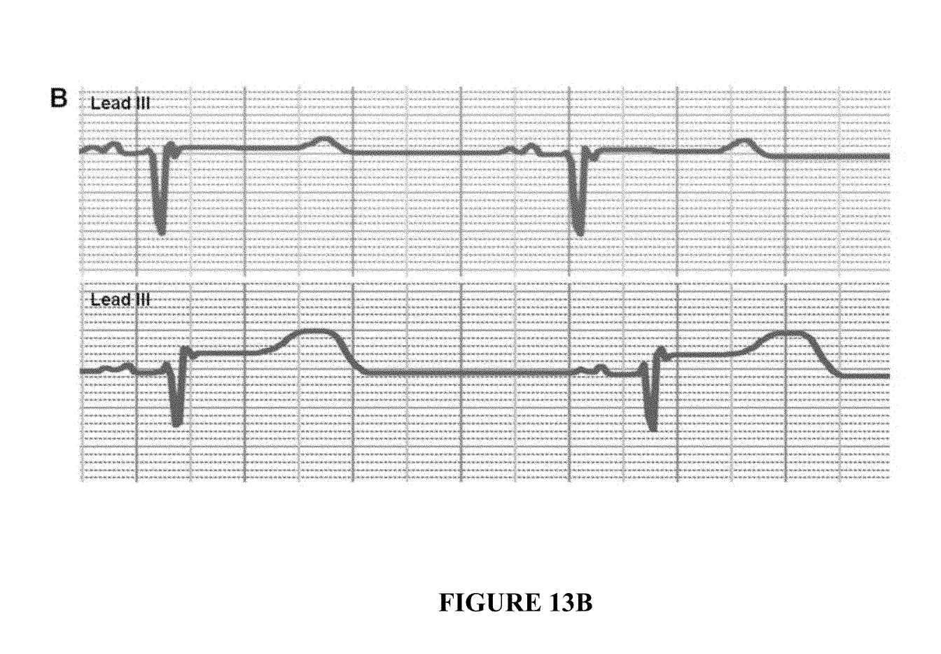

Animals were sedated with telazol [8 mg/kg, i.m.], intubated and ventilated. General anesthesia consisted of isoflurane [1-2%, i.n.]. A 12-lead electrocardiogram (ECG) and arterial pressure were monitored. Left femoral arterial access was obtained and an Amplatz-type catheter was guided into the left main coronary artery under fluoroscopy. A 3 mm angioplasty balloon catheter was then advanced and inflated at the third diagonal coronary artery that arose from the left anterior coronary descending (LAD) artery. Thirty seconds after balloon inflation, a five mL suspension of saline containing one mL polystyrene microspheres (Polysciences Inc., Polybead 90 μm diameter, Warrington, Pa., USA) was injected distally into that artery. Occlusion of the artery was visualized by contrast angiography, and acute MI was confirmed by the presence of ST-segment elevations in lead II or III of the ECG (FIG. 13).

### Experimental Protocol Post-Myocardial Infarction

Healed MI animals were studied 42±2 days post-MI. MI and age-matched control animals were sedated with telazol [8 mg/kg, i.m.], intubated and ventilated. General anesthesia was maintained with isoflurane [1-2%, i.n.]. Depth of anesthesia was monitoring by hemodynamic indices, jaw tone, and pedal withdrawal reflex; anesthesia was adjusted as necessary. Right femoral venous access was obtained for fluid replacement and right femoral arterial access for monitoring arterial pressure. A median sternotomy was performed to expose the heart, as well as stellate ganglia, the IVC, and the descending thoracic aorta. A lateral ventral incision of the neck was performed to expose cervical vagi and carotid arteries. Snare occluders were placed around the vessels (IVC, aorta, and carotid arteries) and stimulating electrodes placed around (vagus) or into (stellate ganglia) autonomic efferent neural structures. Following the completion of surgery, general anesthesia was changed to α-chloralose [50 mg/kg, i.v. bolus with continuous infusion 10 mg/kg/hr, i.v.]. Body temperature was monitored and maintained via heating pads. Acid-base status was evaluated hourly; respiratory rate and tidal volume were adjusted and bicarbonate was infused as necessary to maintain blood gas homeostasis. At the completion of the experiments, animals were humanely euthanized under deep anesthesia using sodium pentobarbital (100 mg/kg, i.v.) followed by potassium chloride (150 mg kg⁻¹, i.v.) to arrest the heart.

### Recording Intrinsic Cardiac Neural Activity

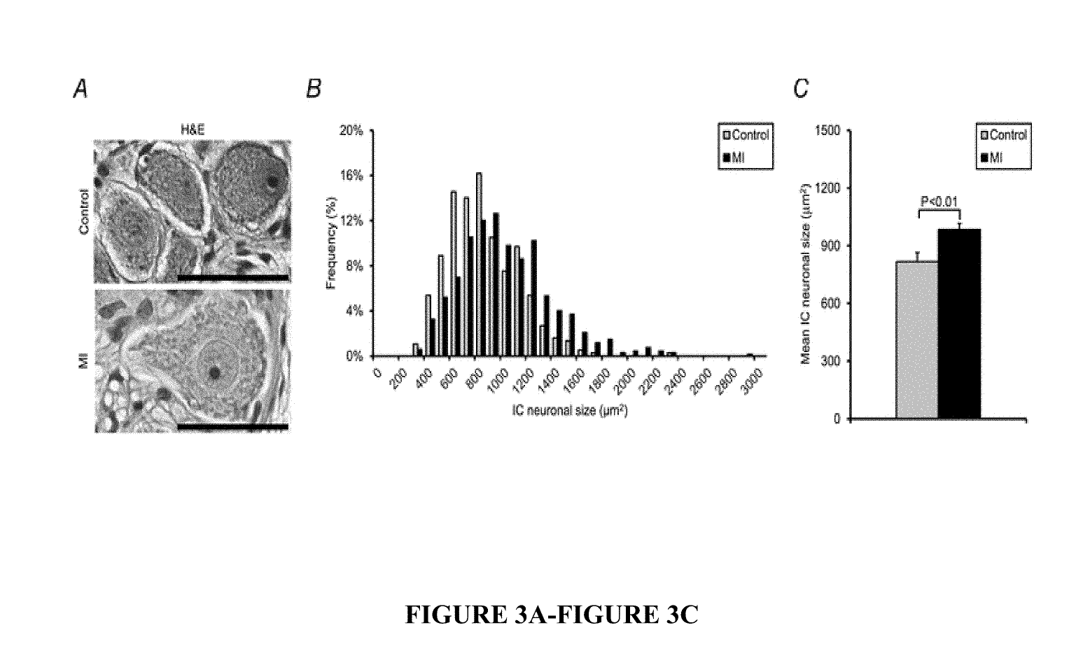

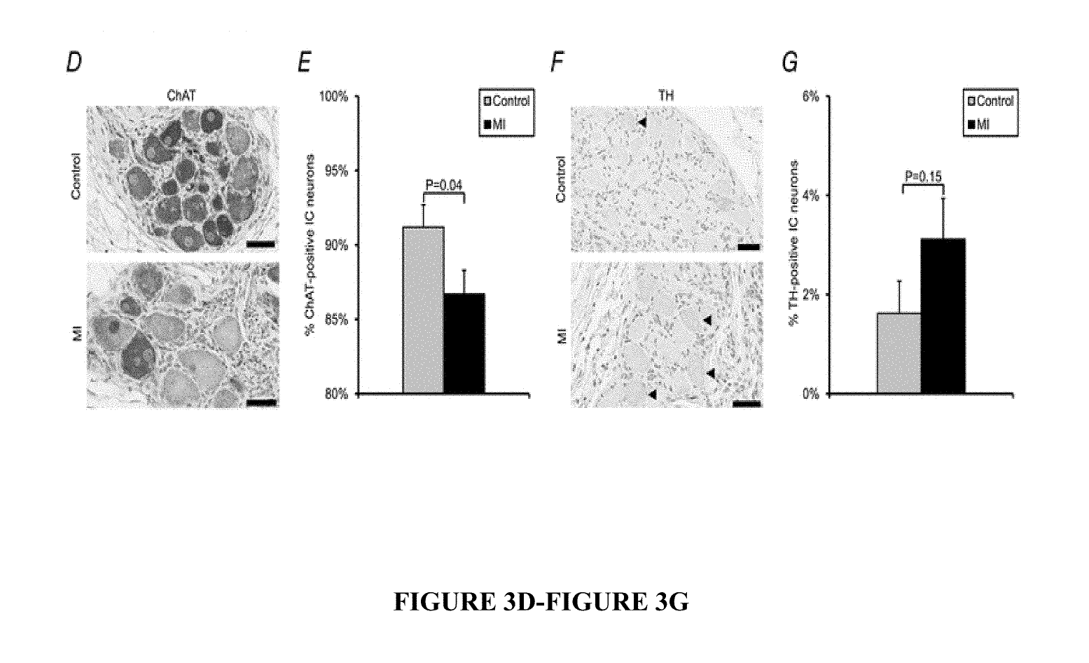

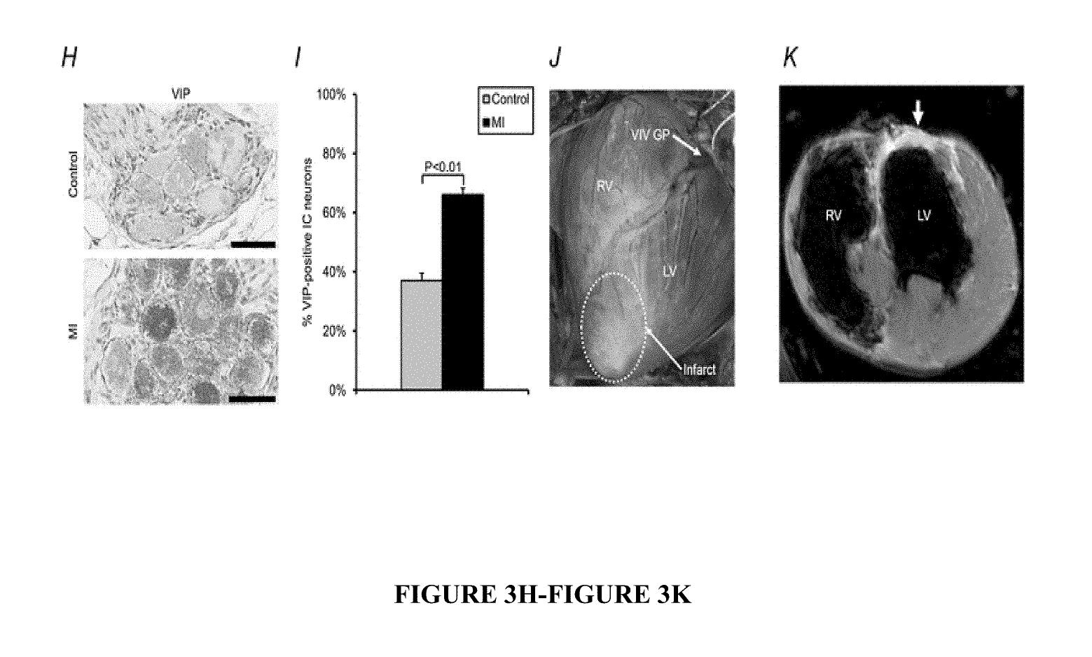

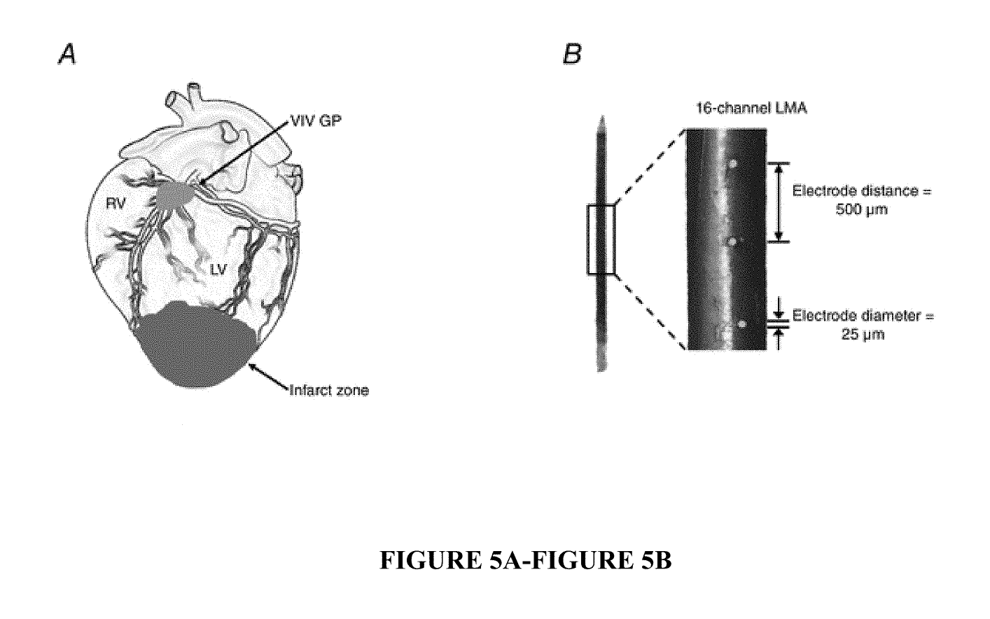

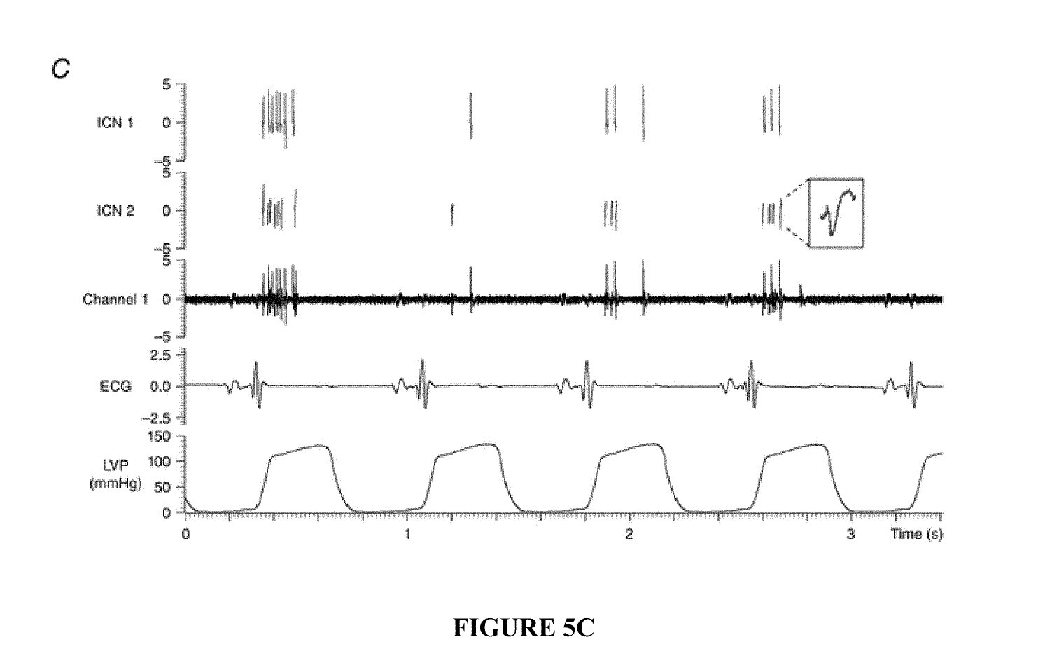

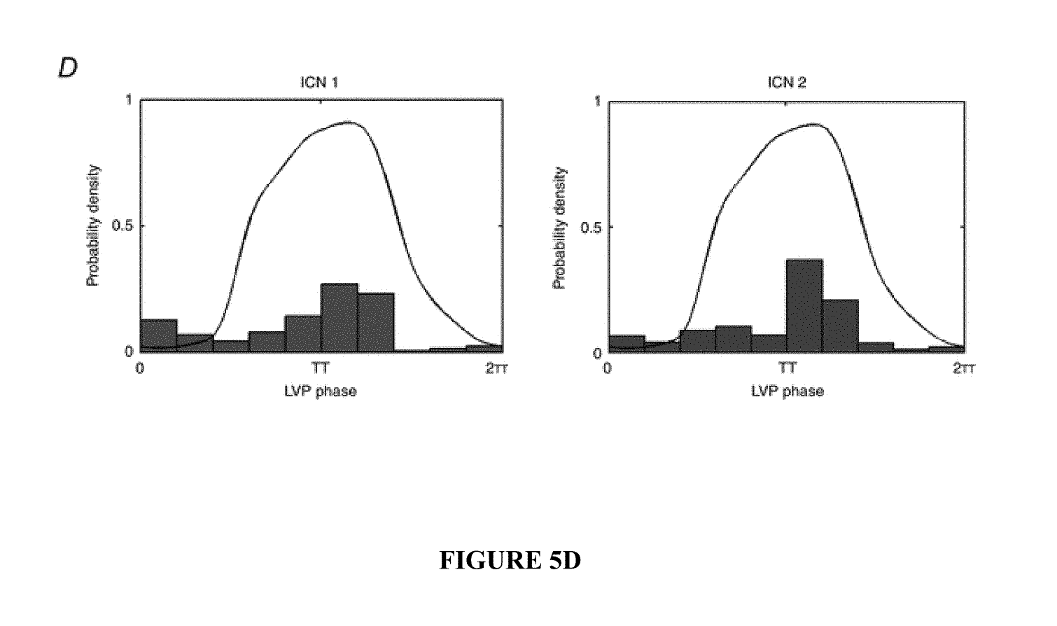

A linear microelectrode array (LMA) (MicroProbes, Gaithersburg, Md., USA) was used to record the in vivo activity generated by neurons in the VIV GP. The LMA consisted of 16 platinum/iridium electrodes (25 μm diameter electrodes with an exposed tip of 2 mm; impedance 0.3-0.5 MΩ at 1 kHz) (FIG. 5B). The electrode was embedded in the VIV GP, which lies near the origin of the LAD from the left main coronary artery (FIG. 3J and FIG. 5A). The LMA was attached to a flexible cable, thereby allowing it to be semi-floating. The electrode wires, as well as earth and reference electrodes, were connected to a 16-channel microelectrode amplifier with a headstage pre-amplifier (A-M Systems Inc., Model 3600, Carlsborg, Wash., USA). For each channel, filters were set to 300 Hz to three kHz with a gain to 5,000. An electrode was sewn to the right atrial myocardium to provide a reference right atrial electrogram (RAE). Neural waveform, electrocardiogram, RAE, and hemodynamic data were input to a data acquisition system (Cambridge Electronic Design, Power1401, Cambridge, UK). Data was analyzed offline using the software Spike2 (Cambridge Electronic Design), as previously described (Beaumont et al., 2013, The Journal of physiology.; 591(Pt 18):4515-33).

### Left Ventricular Hemodynamic Assessment

A pressure catheter (Millar Instruments, Mikro-Tip, Houston, Tex., USA) was placed into the LV chamber via the left femoral artery and connected to a control unit (Millar Instruments, PCU 2000). LV systolic function was evaluated by end-systolic pressure and maximum rate of pressure change (dP/dt maximum). LV diastolic function was evaluated by end-diastolic pressure and minimal rate of chamber pressure change (dP/dt minimum).

### Afferent Neural Input Assessment

In order to determine the capacity of IC neurons to transduce mechanosensory afferent inputs, epicardial mechanical stimuli (gentle touch) were applied for 10 seconds at the following four sites: RVOT, RV apex, LV mid-anterior wall, and LV apex. Transient (30 s) occlusions of the IVC and aorta were then performed using a snare occluder in order to determine the capacity of IC neurons to transduce acute changes in preload and afterload, respectively.

### Efferent Neural Input Assessment

In order to determine which IC neurons receive parasympathetic and sympathetic efferent preganglionic inputs, bipolar spiral cuff electrodes (Cyberonics Inc., PerenniaFlex Model 304, Houston, Tex., USA) were placed around the cervical vagi and bipolar needle electrodes inserted into the stellate ganglia bilaterally. A stimulator with an isolation unit (Grass Technologies, S88 and PSIU6, Warwick, R.I., USA) was used to modulate efferent inputs to IC neurons. For each vagus, threshold was defined as the current necessary to evoke a 10% decrease in heart rate or BP (20 Hz frequency, 1 ms pulse width). For each stellate ganglia, threshold was defined as the current necessary to evoke a 10% increase in heart rate or BP (4 Hz frequency, 4 ms pulse width). Each vagus and stellate ganglion was then stimulated individually for one minute at a current of 1.2 times threshold and at a frequency of one Hz. This was done in order to assess direct inputs to the ICNS independent of any changes in cardiac function. Transient (1 min) occlusion of the bilateral carotid arteries (caudal to carotid sinus) was then performed using a snare occluder to determine the capacity of the carotid baroreflex to modulate efferent inputs to IC neurons.

### Epicardial Pacing

In order to determine the capacity of IC neurons to respond to cardiac electrical stimulation, a bipolar pacing electrode (St. Jude, St. Paul, Minn., USA) was placed at various epicardial sites and pacing (6 mA current; 2 ms pulse width) was performed at 10% above baseline heart rate for 10 captured beats. The following four sites were paced: 1) right atrial appendage, 2) RV outflow tract, 3) RV apex, and 4) LV apex.

### Ventricular Tachyarrhythmia Inducibility

In a separate set of control (n=8) and healed anteroapical MI animals (n=8), ventricular tachyarrhythmia (VT) inducibility was evaluated by programmed ventricular stimulation (EPS320; Micropace, Canterbury, New South Wales, Australia) at two different cycle lengths (600 and 400 ms) with up to three extra stimuli (200 ms minimum) from two different sites (RV apex and LV anterior wall epicardium).

### Tissue Processing

Following completion of IC neural recording, animals were sacrificed and hearts immediately excised. The fat pads containing the VIV GP, dorsal interventricular ganglionated plexus (DIV GP), right marginal artery ganglionated plexus (RMA GP), and right atrial ganglionated plexus (RA GP) were removed, rinsed in cold (4° C.) saline, and transferred to cold 10% phosphate-buffered formalin (Fisher Scientific, Pittsburgh, Pa., USA) for four days. Afterwards, the tissue was transferred to 70% ethanol (Sigma-Aldrich, St Louis, Mo., USA) and paraffin embedded within three days. Four μm thick sections were cut from the paraffin blocks.

### Histologic Staining

IC neuronal size was determined from hematoxylin and eosin (H&E) stained sections (Fisher Scientific, PROTOCOL, Pittsburgh, Pa., USA) using computerized morphometric analysis (Aperio ImageScope, Leica Biosystems, Buffalo Grove, Ill., USA).

### Immunohistochemical Stains

IC neuronal adrenergic phenotype was quantified by tyrosine hydroxylase (TH) immunoreactivity (1:2000 dilution, Abcam, #ab112, Cambridge, Mass., USA); neuronal cholinergic phenotype by ChAT immunoreactivity (1:200 dilution, Millipore, AB144-P, Billerica, Mass., USA); and vasoactive intestinal peptide (VIP) immunoreactivity by anti-VIP Ab (ImmunoStar, Catalog #20077, Hudson, Wis., USA). Secondary detection was performed with Dako EnVision+ System− HRP Labeled Polymer Anti-Rabbit (Dako North America Inc., K4003, Carpinetria, Calif., USA) for TH and VIP at 1:500, and Polyclonal Rabbit Anti-Goat Immunoglobulins/Biotinylated (Dako, E0466) for ChAT. Secondary immunoreactivity was detected by diaminobenzidine (Life Technologies, Green Island, N.Y., USA) per manufacturer's recommended protocol for all stains. The slides were then scanned digitally and analyses performed on the electronic images. All neurons present in the slides were quantified using computerized image analysis (Aperio ImageScope) at 20-40× magnifications. Staining and quantification of the groups were performed in a blinded fashion.

### Data Analysis: Signal Processing of Multi-Unit Intrinsic Cardiac Neural Activity

Artifact removal and IC neuronal identification was performed using off-line analysis (FIG. 5C). Briefly, recorded IC neuronal activity was contaminated by endogenous electrical artifact arising from the activity of the adjacent atrial and ventricular myocardium, as well as by exogenous electrical artifact arising from stimulation of autonomic efferent nerves. Simultaneously occurring activity displaying similar waveforms in more than 3 adjacent channels of the LMA was also considered to be artifact. After identification, artifacts were removed from all channels by blanking. This process resulted in a maximum loss of 3% of the total signal. Following artifact removal, individual units were sorted using principal component analysis of waveform shapes (Beaumont et al., 2013, The Journal of physiology.; 591(Pt 18):4515-33).

### Data Analysis: Monitoring the Activity of Individual Intrinsic Cardiac Neurons

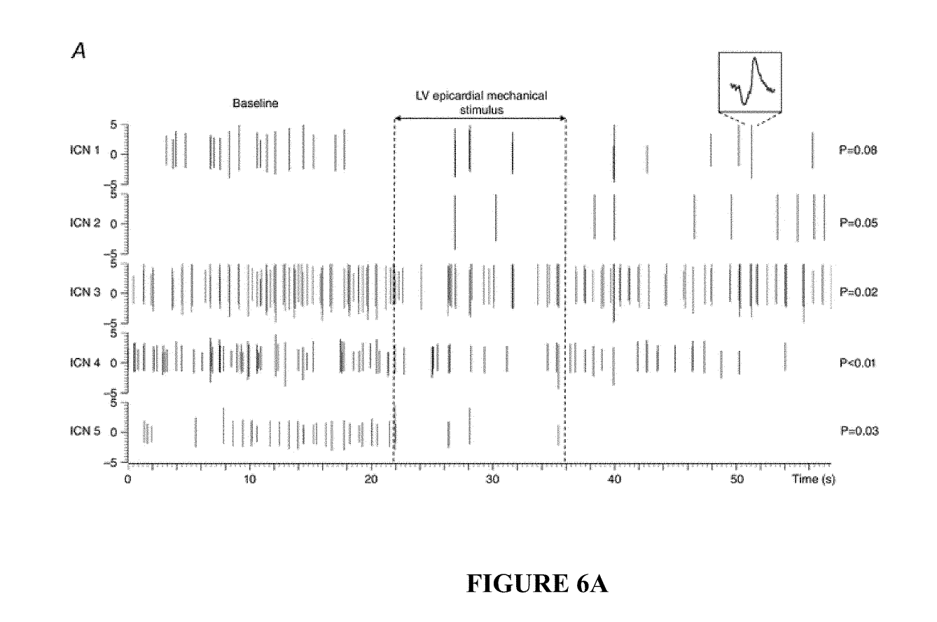

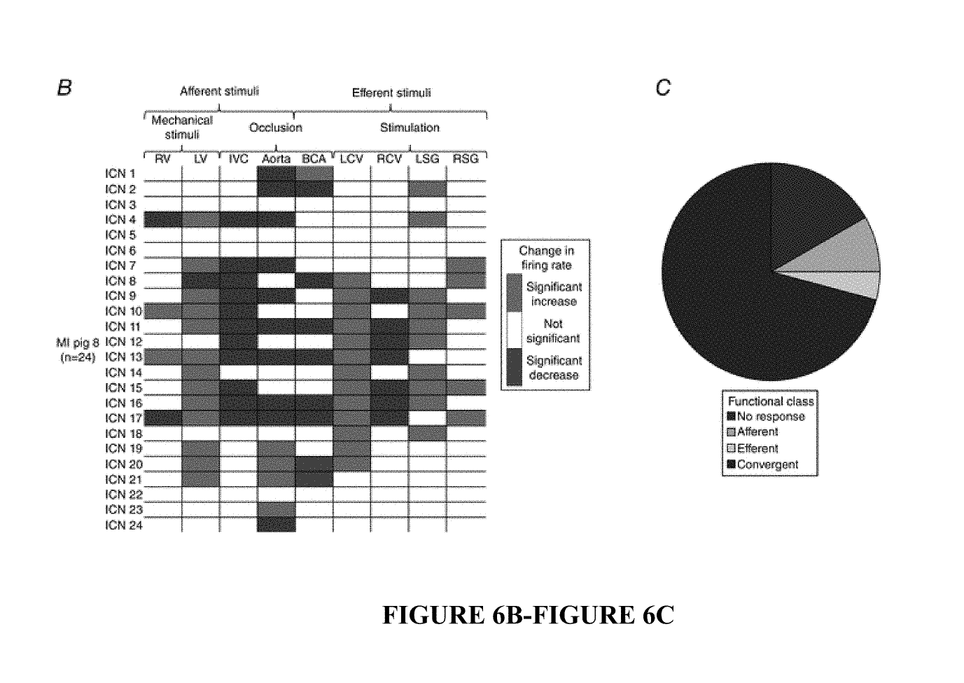

For epicardial mechanical stimuli and autonomic efferent nerve stimulations, IC neuronal activity was compared one minute before the stimuli (baseline) versus during the stimuli. For vascular occlusions and pacing, IC neural activity was compared at baseline versus during the stimuli, as well as at baseline versus one minute after the stimuli (recovery). After each stimulus, a waiting period of at least five minutes was taken for IC neural activity and hemodynamics to return to baseline levels before proceeding. IC neurons were functionally classified as afferent, efferent or convergent based on their response characteristics to the cardiovascular stimuli (FIG. 6B and FIG. 6C). Afferent IC neurons were defined as those that responded solely to epicardial mechanical stimuli and/or occlusion of the IVC or aorta. Efferent IC neurons were defined as those that responded solely to stimulation of autonomic efferent nerves (vagus or stellate ganglia) and/or occlusion of the bilateral carotid arteries. IC neurons that responded to activation of both afferent and efferent inputs were defined as convergent. (Beaumont et al., 2013, The Journal of physiology.; 591(Pt 18):4515-33).

### Data Analysis: Conditional Probability

Conditional probability analysis was used to determine whether an IC neuron that responded to one stimulus also responded to another stimulus, as previously described (Beaumont et al., 2013, The Journal of physiology.; 591(Pt 18):4515-33). The potential for a functional relationship between stimulus X and stimulus Y was quantified within neurons identified in each animal as a conditional probability that a neuron that responded to stimulus Y also responded to stimulus X. The conditional probability (probability: response to Y|response to X) was estimated as the number of neurons that responded to both stimulus X and stimulus Y, divided by the number of neurons that responded to stimulus X.

### Statistics

The significance level of changes in the firing rate of each IC neuron between baselines versus stimulus/recovery intervals was assessed using a statistical test developed for cortical neurons based on the Skellam distribution. A chi-square test was used to compare the IC neural response in MI versus control animals. A Wilcoxon signed-rank test or Mann-Whitney U test was used to compare IC neural firing frequencies, resting hemodynamic indices, as well as morphologic and phenotypic changes in IC neurons in MI versus control animals. Data are presented as mean±standard error. A p value of less than 0.05 was considered to be statistically significant. Statistical analyses were performed using SigmaPlot 12.0 (Systat Software Inc., San Jose, Calif., USA).

### Results

Healed anteroapical MI animals were in a chronic compensated state and not in overt heart failure, as evidenced by the lack of change in resting hemodynamic indices such as left ventricular (LV) end-diastolic pressure (3±1 mmHg in MI versus 4±1 mmHg in control (p=0.59)), and LV dP/dt maximum (1436±112 mmHg/s in MI versus 1426±151 mmHg/s in control (p=1.00)). FIG. 3J-FIG. 3K illustrates a typical pattern of scar formation induced by the microembolization technique.

### Myocardial Infarction Induces Intrinsic Cardiac Neural Enlargement and Phenotypic Changes

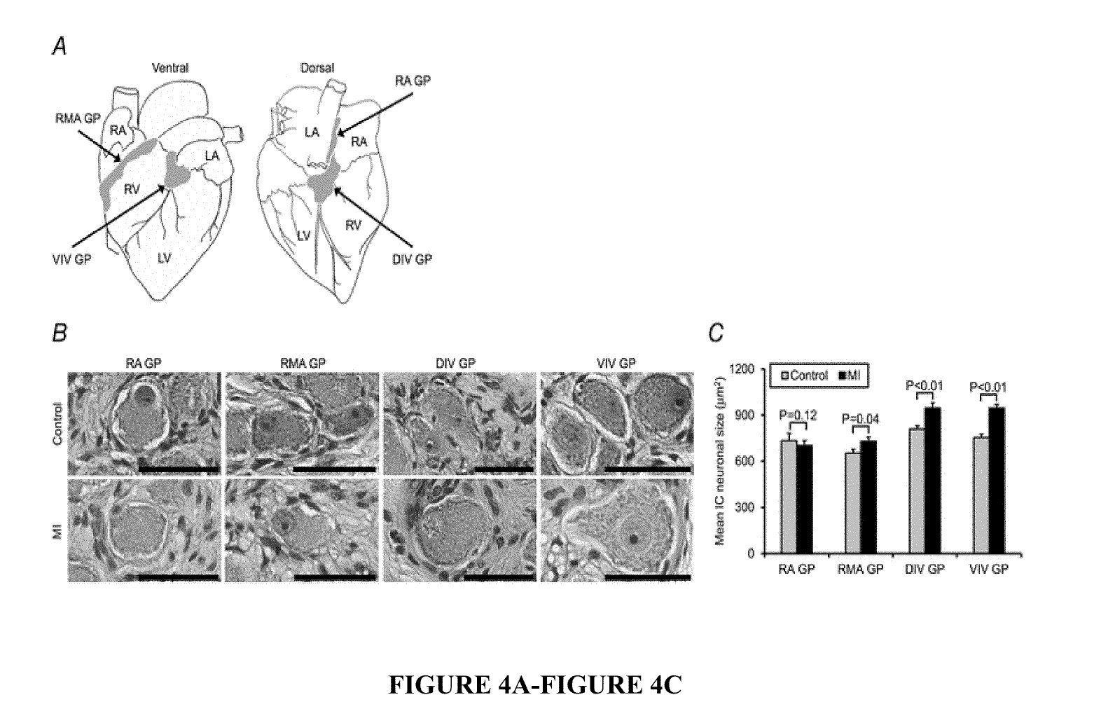

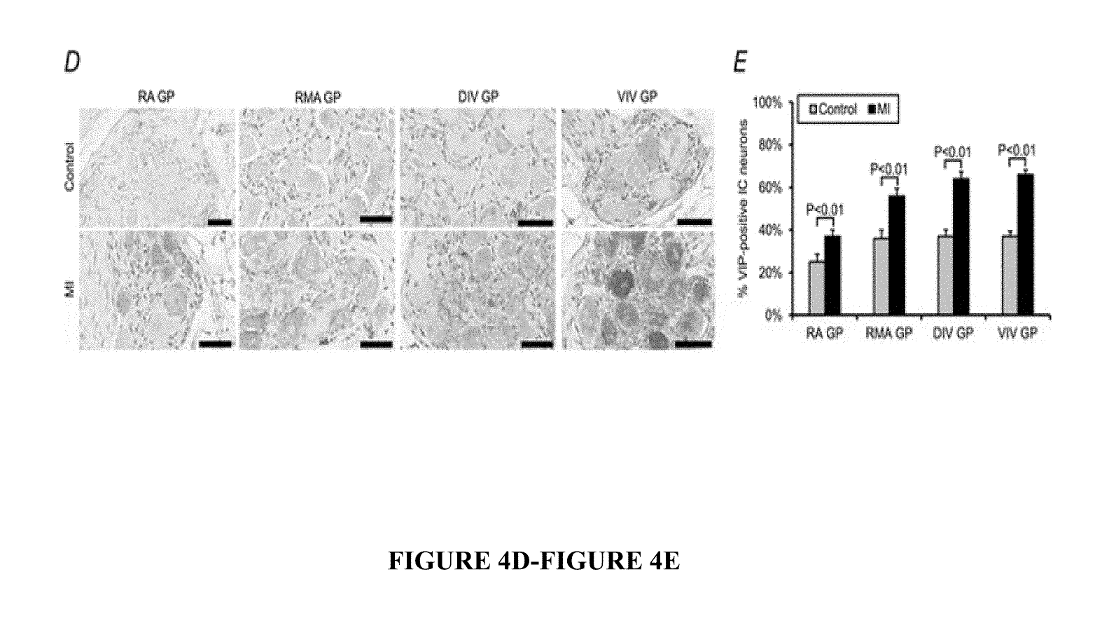

Neurons from the ventral interventricular ganglionated plexus (VIV GP) were subjected to histologic and immunohistochemical analyses for size, adrenergic-cholinergic phenotype, and vasoactive intestinal peptide (VIP) phenotype in order to evaluate for morphologic and neurochemical changes induced by MI. VIP is a modulator of cardiac function and a potential afferent marker. VIV GP neurons from MI animals were larger than those from controls (946±23 μm² versus 755±22 μm², respectively; p<0.01) (FIG. 3A-FIG. 3C). A histogram of neural size distribution from MI and control animals is shown in FIG. 3B. Neuronal enlargement was observed in the VIV GP, dorsal interventricular ganglionated plexus, and right marginal artery ganglionated plexus, which exert preferential influence over the ventricles, but was not observed in the right atrial ganglionated plexus, which exerts preferential influence over the atria (FIG. 4A-FIG. 4C).

There was a significant decrease in the percentage of IC neurons expressing choline acetyltransferase (ChAT) in MI animals compared to controls (87±2% versus 91±2%, respectively; p=0.04) (FIG. 3D-FIG. 3E). In contrast, there was no difference in tyrosine hydroxylase (TH) expression in MI relative to control animals (3±1% versus 2±1%, respectively; p=0.15) (FIG. 3F-FIG. 3G). VIP expression was increased in MI versus control animals (66±2% versus 37±3%, respectively; p<0.01) (FIG. 3H-FIG. 3I). VIP expression was also significantly increased in all the other ganglionated plexi (GPs) studied (FIG. 4D and FIG. 4E).

### Functional Characterization of Intrinsic Cardiac Neurons Post-Myocardial Infarction

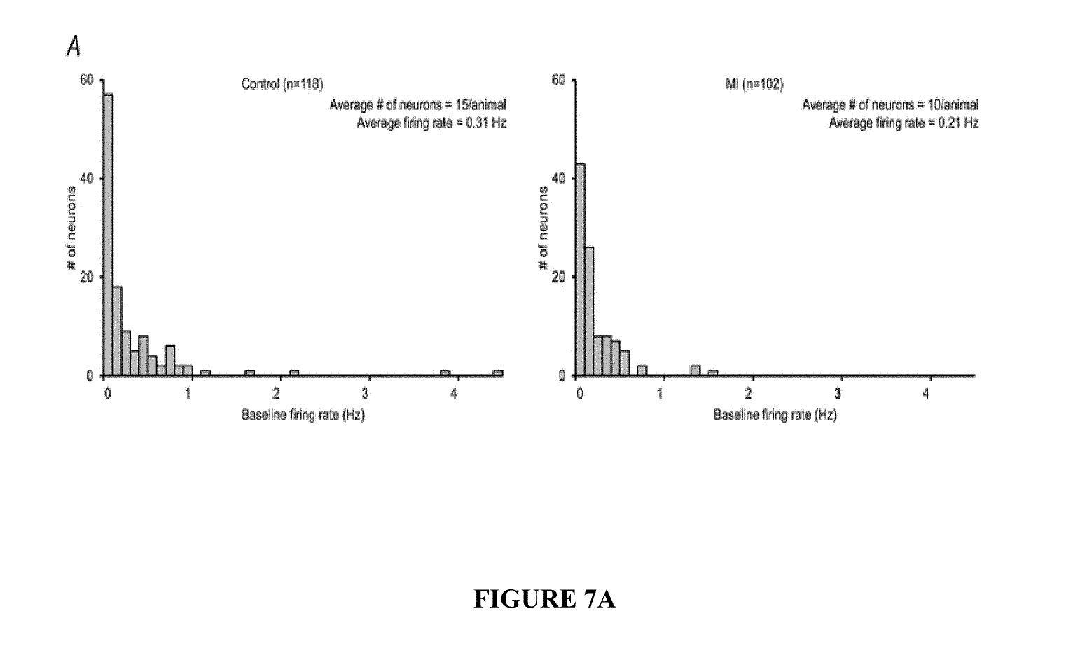

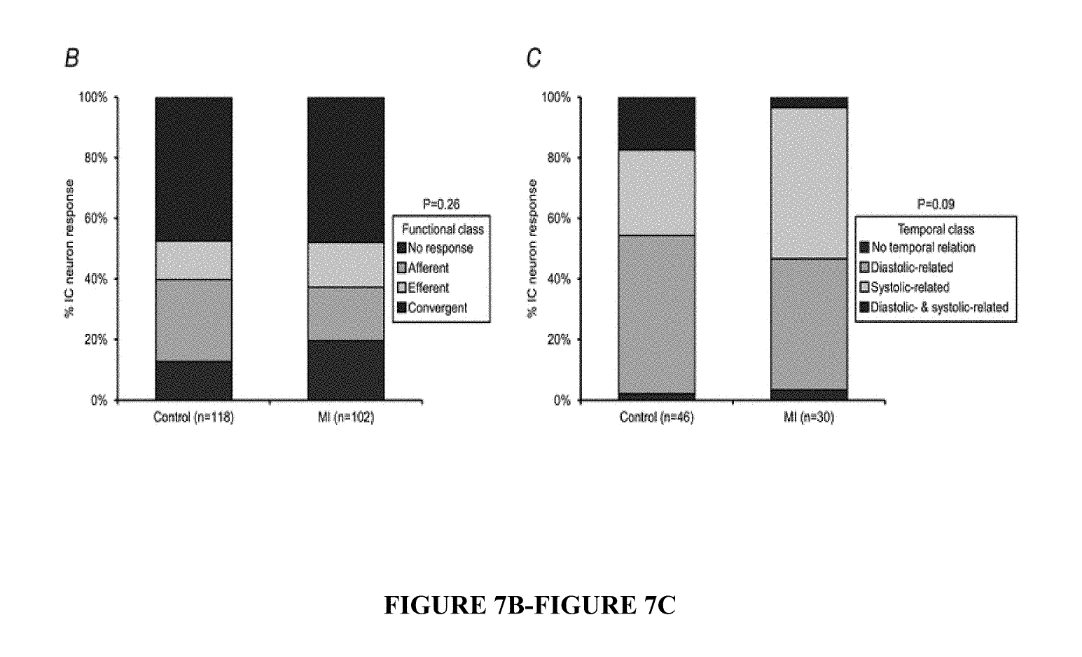

The in vivo activity of neurons from the VIV GP was recorded in control and MI animals using a linear microelectrode array (LMA) in order to evaluate functional changes in neural response characteristics induced by MI (FIG. 5 and FIG. 6). In 8 control animals, the activity generated by 118 IC neurons from the VIV GP was studied (average: 15±3 neurons per animal) (FIG. 7A, left panel). In 8 MI animals, the activity generated by 102 neurons was studied (average: 10±2 neurons per animal) (FIG. 7A, right panel). The spontaneous firing rates of neurons were derived from pooling data from baseline intervals. The average spontaneous firing rate of the neurons from control animals was 0.31 Hz (range: 0 to 4.42 Hz), while the average of those from MI animals was 0.21 Hz (range: 0 to 1.59 Hz). The distribution was overall similar in both states, with more than 90% of neurons firing below 1 Hz.

Based on their response characteristics to the cardiovascular stimuli, IC neurons were functionally classified as afferent, efferent, or convergent (FIG. 6B, FIG. 6C, and FIG. 7B). In control and MI animals, convergent neurons represented the largest subpopulation (47% versus 48%, respectively), followed by fewer afferent (27% versus 18%, respectively) and efferent (13% versus 15%, respectively) neurons (FIG. 7B). 13% of neurons in control animals and 20% in MI animals did not respond to any of the stimuli. There was no difference in the overall classifications in either state (p=0.26).

The activity of IC neurons was compared to the cardiac cycle in order to determine if they exhibited cardiac cycle-related periodicity (FIG. 5D and FIG. 7C). Based on an activity histogram, neurons that generated at least 100 action potentials at baseline were classified as being related to a specific phase of the cardiac cycle if more than 30% of their activity occurred during the given phase. Forty-six neurons (39%) in control animals and 30 neurons (29%) in MI animals that satisfied this criterion were analyzed for cardiac-related periodicity (FIG. 7C). In control animals, 52% of neurons displayed diastolic-related activity, 28% displayed systolic-related activity, 17% displayed dual diastolic- and systolic-related activity, and 2% displayed stochastic behavior. In contrast, in MI animals, 43% of neurons displayed diastolic-related activity, 50% displayed systolic-related activity, 3% displayed dual diastolic- and systolic-related activity, and 3% displayed stochastic behavior.

### Afferent Remodeling of Intrinsic Cardiac Neurons Post-Myocardial Infarction

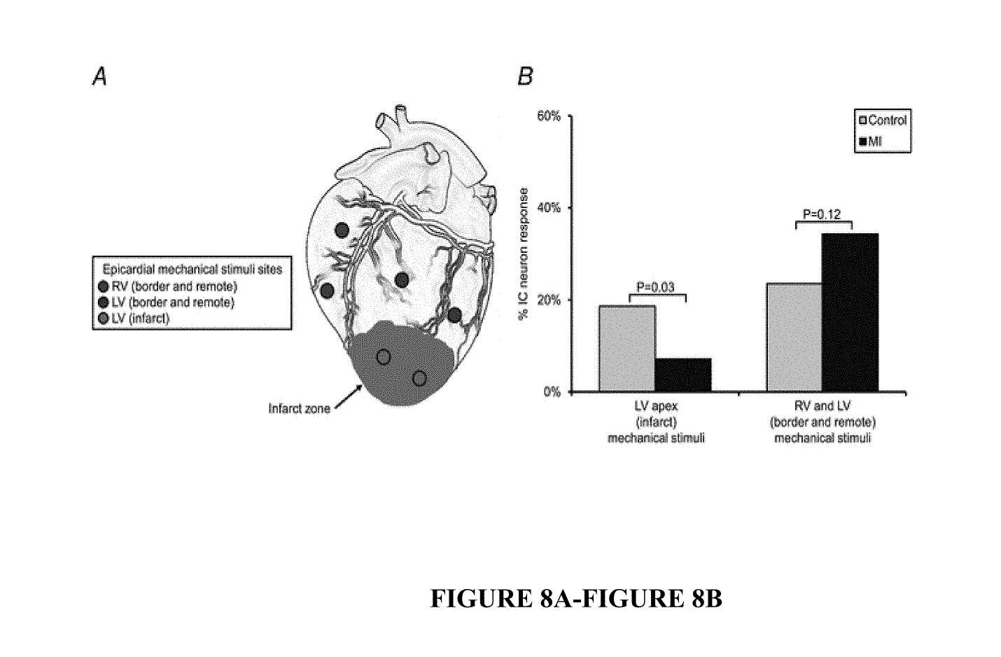

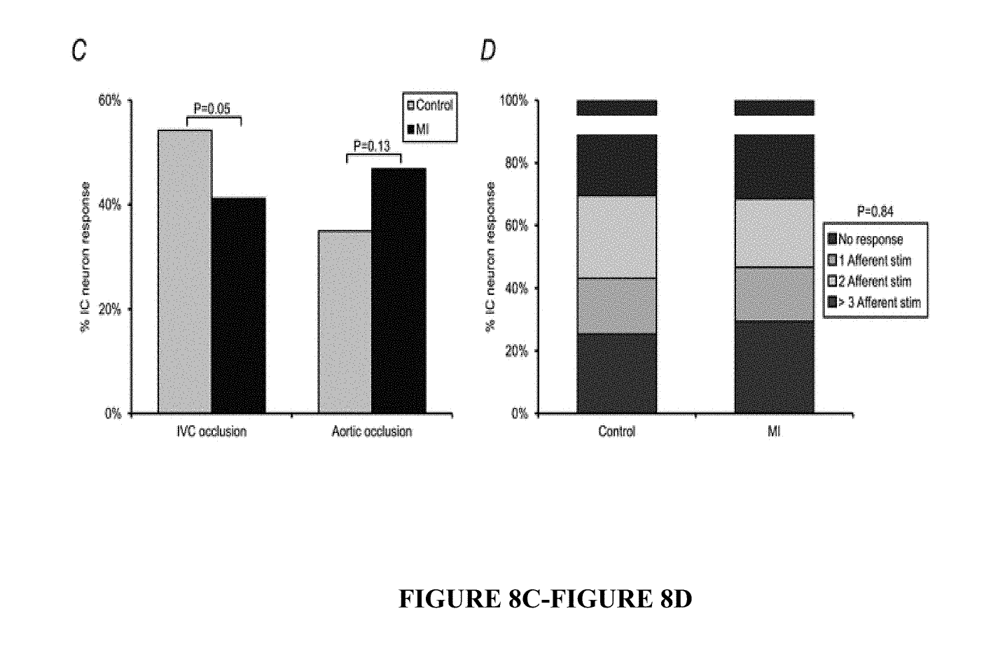

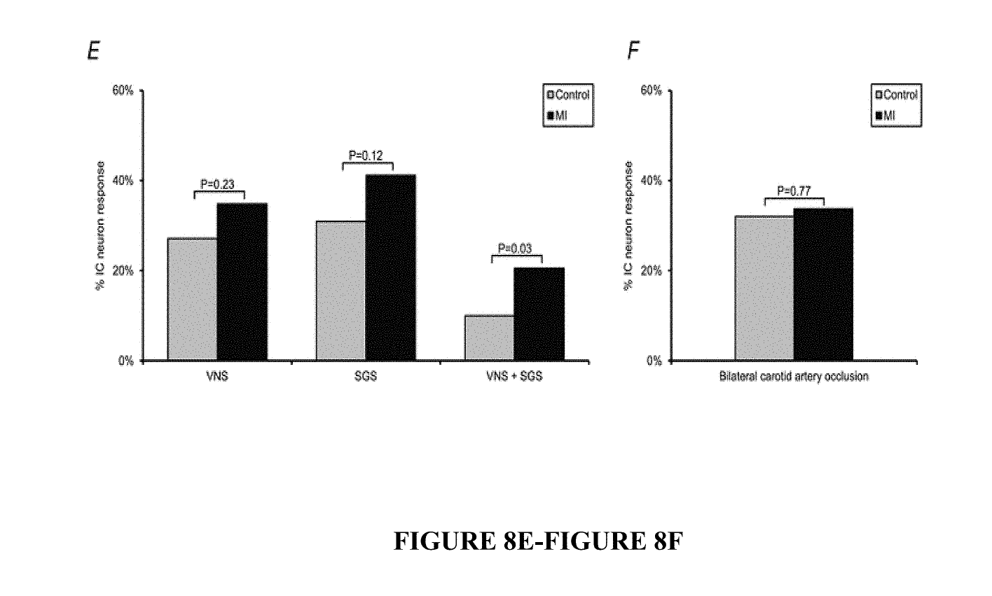

MI differentially affected the capacity of IC neurons to transduce mechanosensitive afferent inputs arising from the infarct versus border and remote zones of the heart, as assessed by applying mechanical stimuli to myocardial tissue overlying these regions (FIG. 8A-FIG. 8B). The neural response to activation of mechanosensitive inputs arising from border and remote zones (RVOT, right ventricular (RV) apex, and LV mid-anterior wall) in MI animals was similar to that in controls (34% in MI versus 24% in control; p=0.12). In contrast, fewer neurons recorded from the VIV GP responded to activation of mechanosensitive inputs arising from the infarct zone (LV apex) following MI (7% in MI versus 19% in control; p=0.03).

The capacity of IC neurons to transduce changes in cardiac loading conditions was also impacted following MI (FIG. 8C). In MI animals, the neural response to a decrease in preload induced by transient inferior vena cava (IVC) occlusion was diminished (41% in MI versus 54% in control; p=0.05). There was no difference in the neural response to an increase in afterload induced by transient occlusion of the descending aorta (47% in MI versus 35% in control; p=0.13). MI likewise did not alter the overall capacity of neurons to transduce multimodal afferent neural signals (FIG. 8D; a similar percentage of neurons in both states received 1, 2, or greater than 3 afferent inputs (p=0.84)). These afferent inputs included epicardial mechanical stimuli and transient occlusion of the IVC and aorta.

### Efferent Remodeling of Intrinsic Cardiac Neurons Post-Myocardial Infarction

MI did not affect the capacity of IC neurons to individually transduce parasympathetic and sympathetic efferent inputs, as assessed by low frequency stimulation of the cervical vagi and stellate ganglia, respectively (FIG. 8E). Stimulation of these autonomic efferent nerves was carried out at low frequencies to evaluate direct efferent inputs to the ICNS, rather than an indirect response resulting from changes in cardiac function. There was no difference in the percentage of neurons receiving inputs from the either the left (LCV) or right cervical vagus (RCV) in MI animals compared to controls (35% versus 27%, respectively; p=0.23). A similar pattern was observed with regards to the percentage of neurons receiving inputs from either the left (LSG) or right stellate ganglion (RSG) (41% in MI versus 31% in control; p=0.12). Interestingly, there was an increase in the percentage of neurons that received efferent inputs from both the sympathetic and parasympathetic divisions of the ANS in MI animals relative to controls (21% versus 10%, respectively; p=0.03). Of these neurons that received both sympathetic and parasympathetic inputs, 90% also responded to activation of one or more afferent inputs in both control (10 of 11 neurons) and MI (19 of 21 neurons) animals. As such, these neurons were classified as convergent.

To assess the effects of MI on baroreflex modulation of efferent inputs to IC neurons, the bilateral carotid arteries were occluded caudal to carotid sinus (FIG. 8F). There was no difference in the percentage of neurons responding to carotid artery occlusion in either state (34% in MI versus 32% in control; p=0.77).

### Myocardial Infarction Induces Changes in Intrinsic Cardiac Neural Response to Pacing

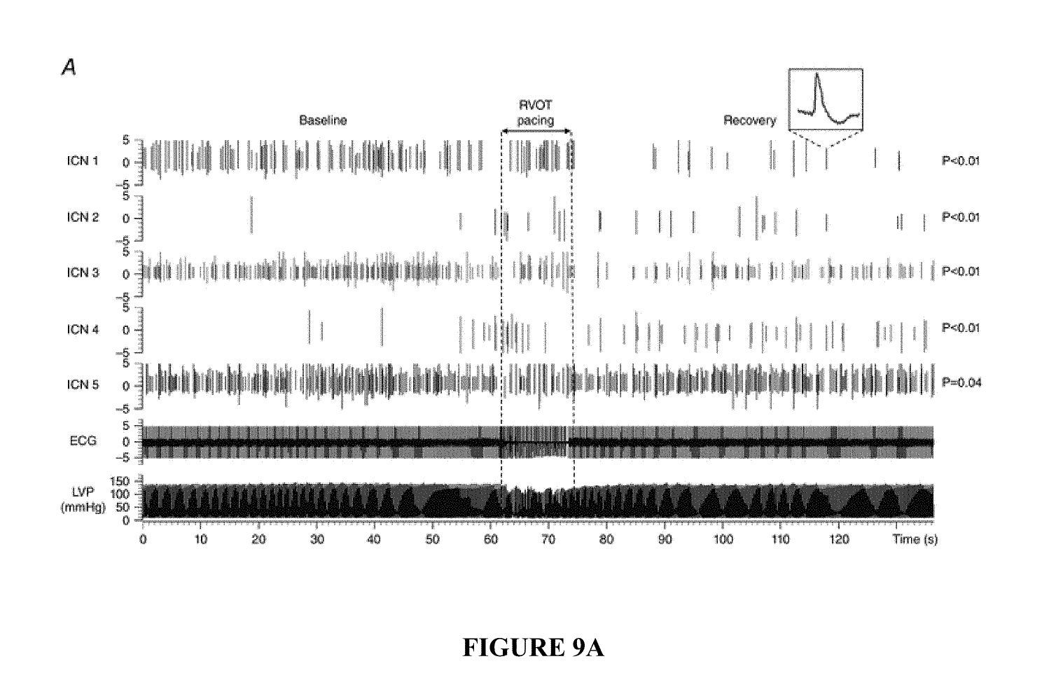

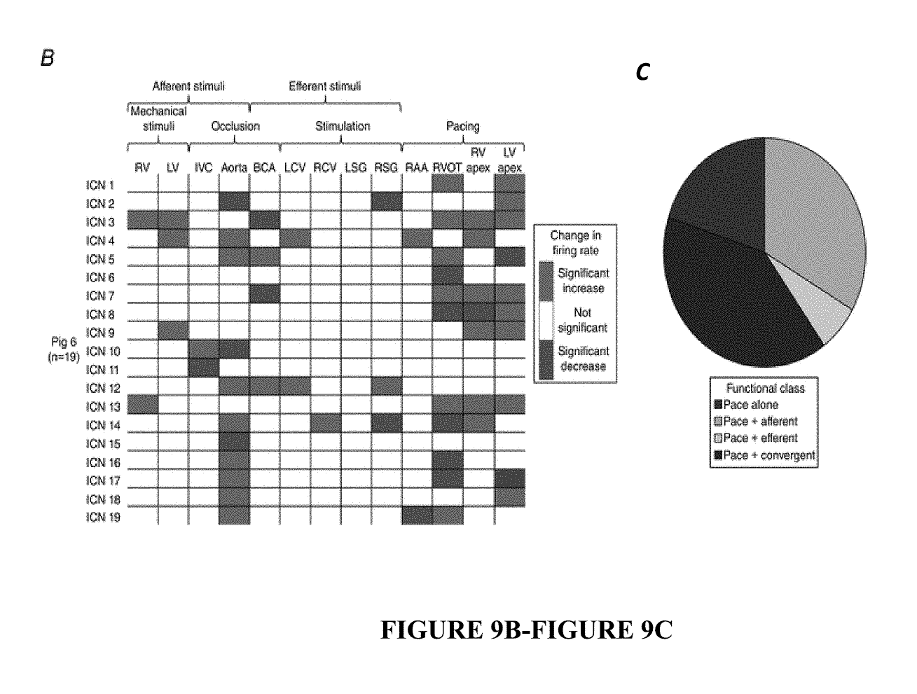

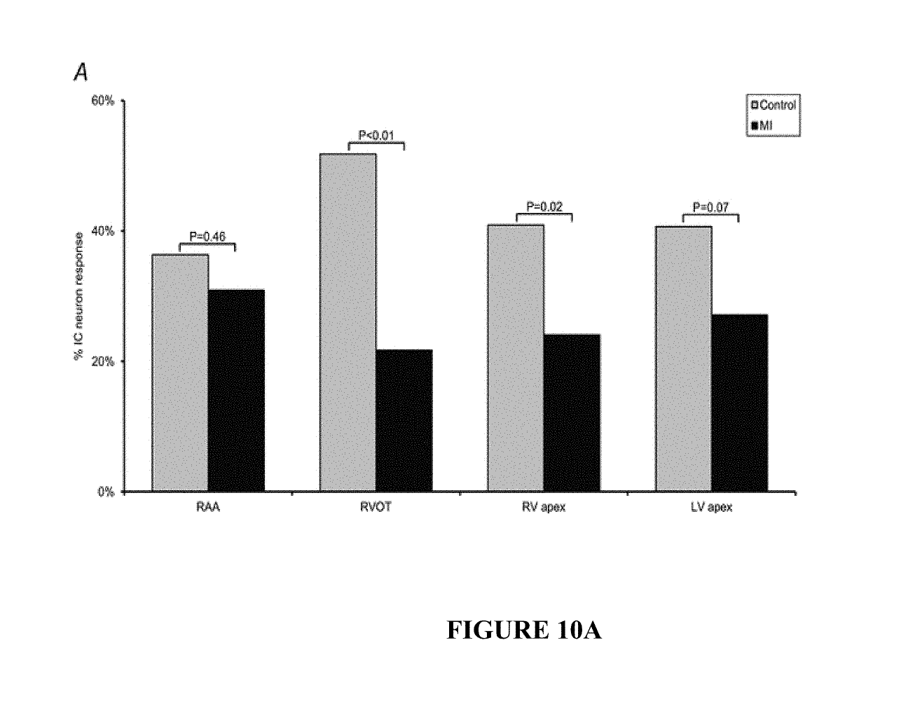

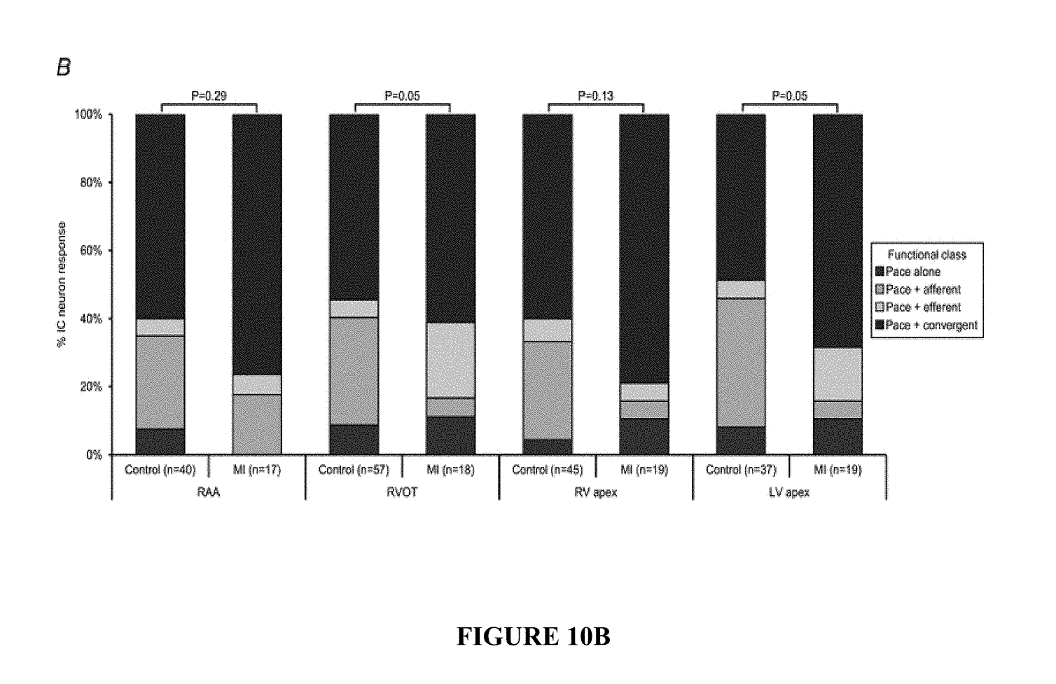

MI differentially impacted the response of IC neurons to epicardial pacing (FIG. 9A and FIG. 10A). Whereas the neuronal response to right atrial appendage (RAA) pacing (with ventricular capture) was not altered (31% in MI vs. 36% in control; P=0.46), the response to ventricular pacing was reduced (44% in MI vs. 63% in control; P<0.01). Neurons that responded to pacing were classified functionally as afferent, efferent, or convergent (FIG. 9A, FIG. 9B and FIG. 10B). The neuronal response evoked from pacing at the right ventricular outflow tract (RVOT) (remote zone) and LV apex (infarct) was most dramatically affected post-MI (p=0.05). This alteration was primarily reflected as an upregulation of pacing-responsive convergent neurons and a corresponding downregulation in pacing-responsive afferent neurons. It is also noteworthy that in all sites evaluated in control animals and most sites in MI animals pacing engaged a unique subpopulation of neurons (FIG. 10B), which only responded to pacing and none of the other afferent or efferent stimuli.

### State Dependence of Intrinsic Cardiac Neurons: Impact on Evoked Response

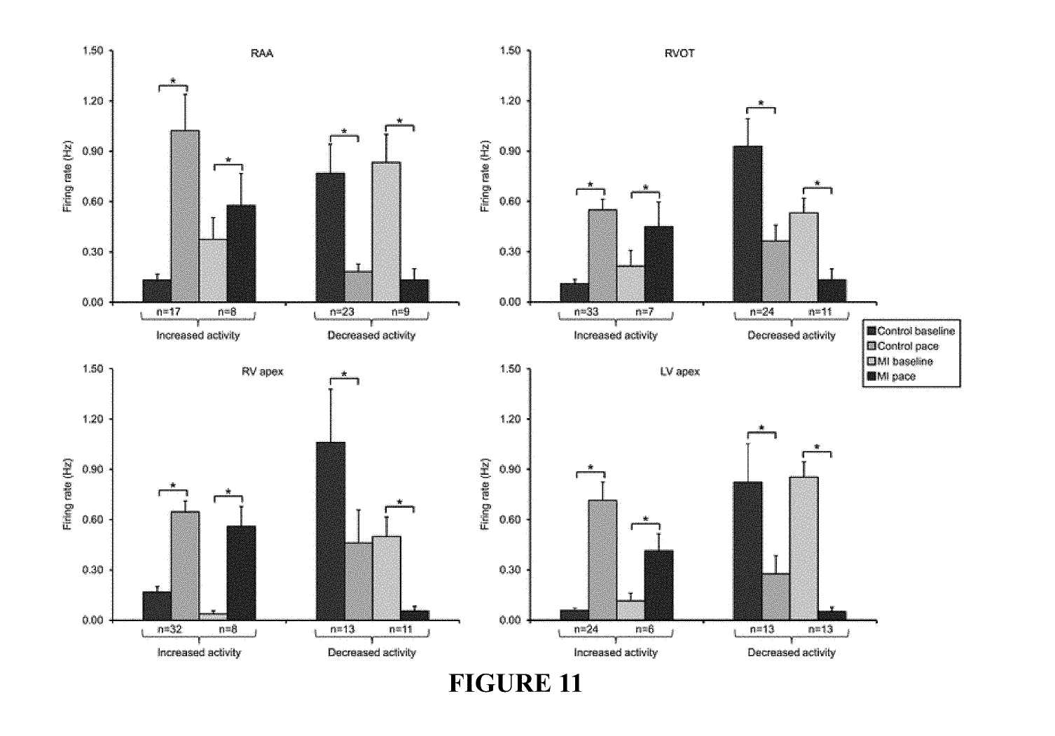

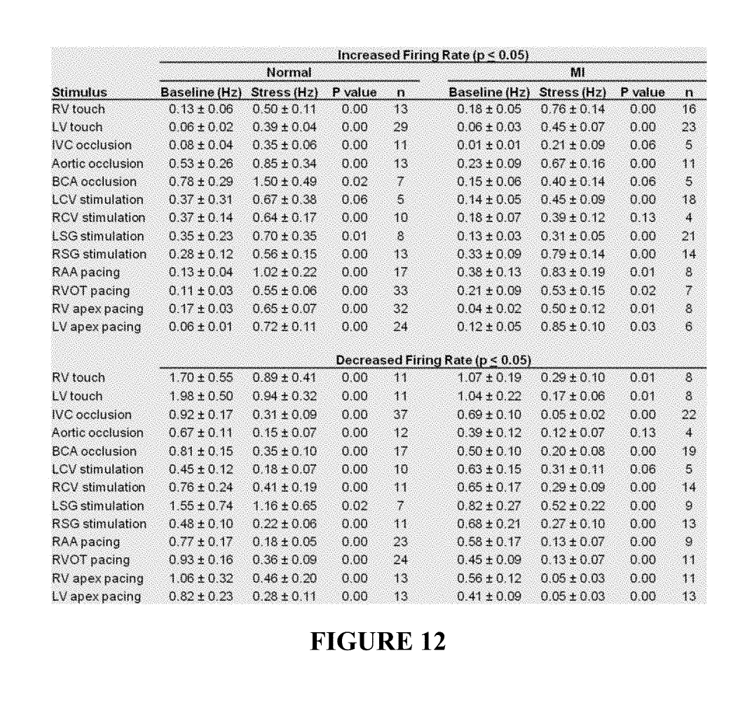

Basal activity impacts IC neural response to subsequent cardiovascular stimuli, including pacing (FIG. 11 and FIG. 12). In both control and MI animals, neurons with low basal activity tended to be activated by pacing (p<0.01). Conversely, neurons with high basal activity tended to be suppressed by pacing (p<0.01). These results indicate the state-dependent nature of such neurons.

### Interdependence of Intrinsic Cardiac Neuronal Response to Stimuli Post-Myocardial Infarction

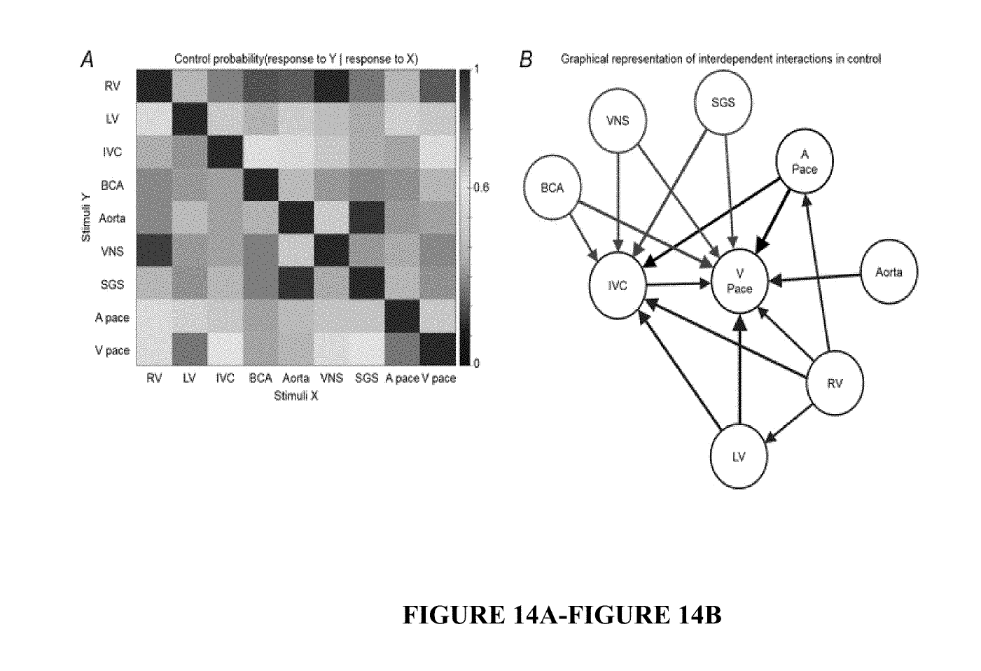

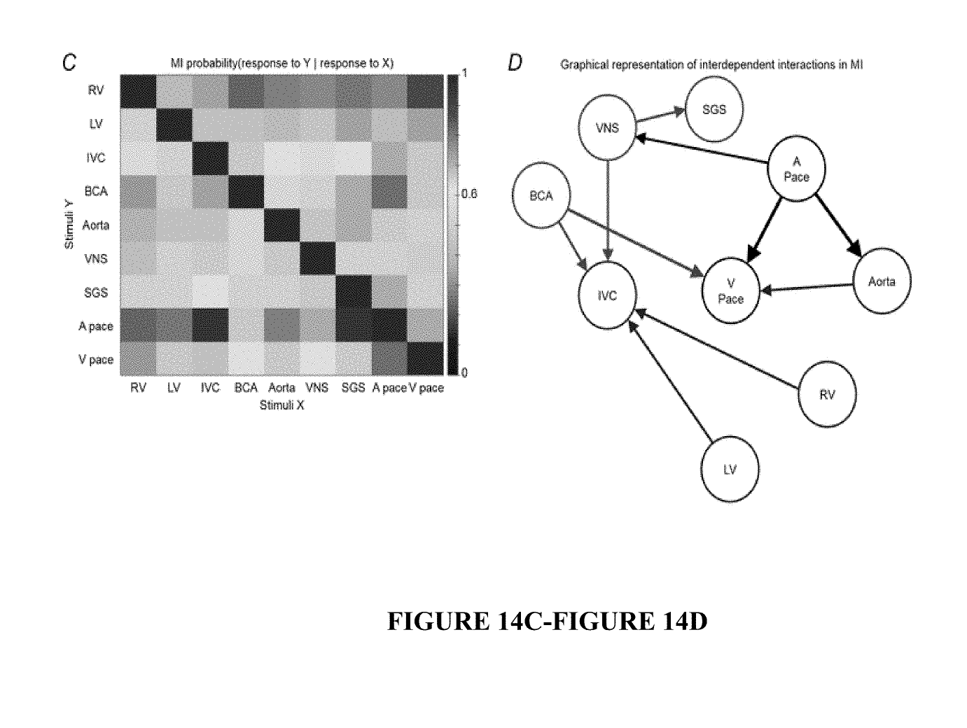

The relationship between the IC neuronal responses to afferent and efferent stimuli, as well as pacing, was determined in both control and MI animals. The conditional probability of whether a neuron that responded to one stimulus also responded to another stimulus is represented in a matrix format (FIG. 14A and FIG. 14C). These data are also depicted graphically as a network, with only links with conditional probabilities≥0.6 displayed (FIG. 14B and FIG. 14D). These relationships reflect the concordant behavior among VIV GP neuronal populations induced by pairs of independent stimuli. MI reduces the overall functional network connectivity within the ICNS.

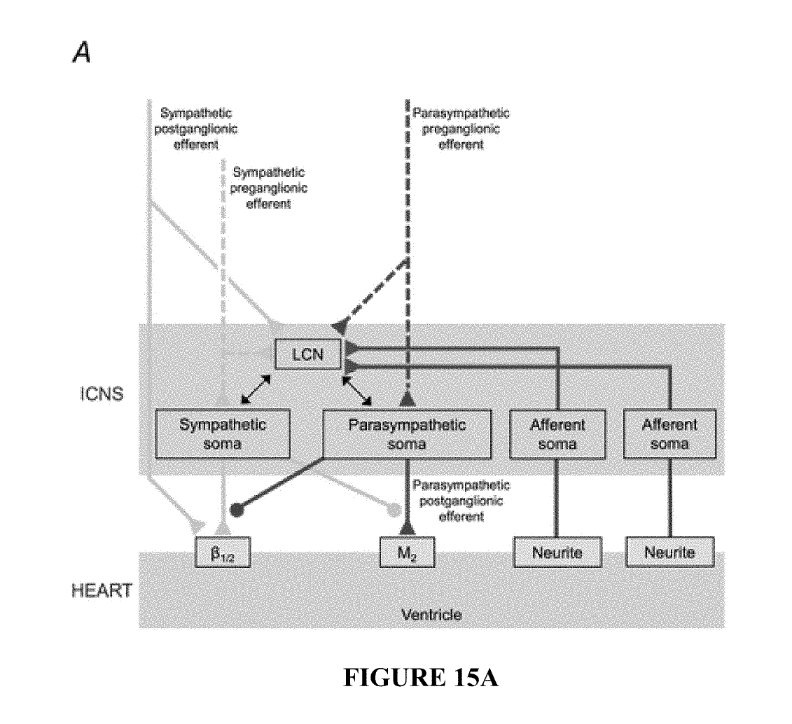

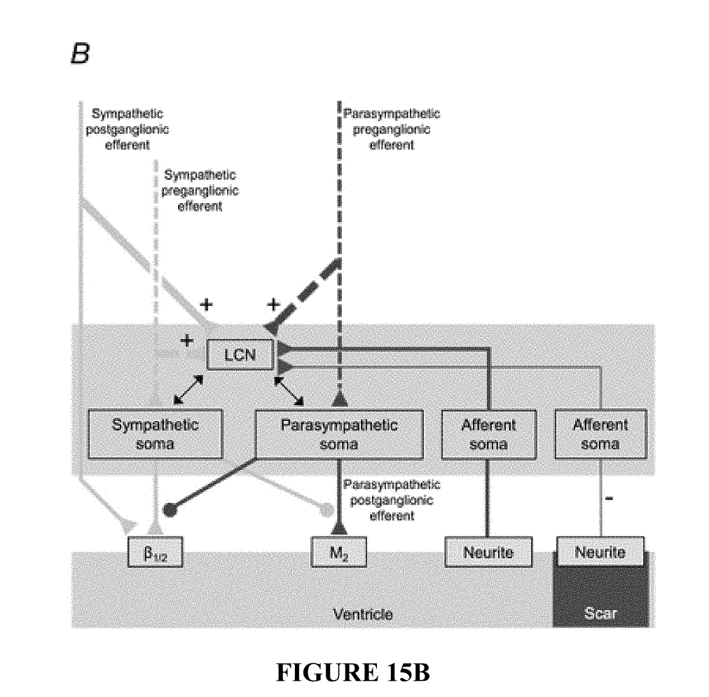

The present study characterized the in vivo structural and functional remodeling of neural elements within the ICNS during the evolution of MI. Neural network function of ICNS was assessed in the VIV GP, a nodal point primarily associated with control of ventricular function. There are several major findings in this study. First, IC neurons differentially enlarge and undergo phenotypic changes post-MI. The site of injury determines which ganglia remodel. Second, afferent neural signaling from the infarcted region to IC neurons are attenuated, while those from border and remote regions are preserved following MI, giving rise to a ‘neural sensory border zone’, or heterogeneity in afferent information from injured vs. adjacent non-injured myocardial tissue (FIG. 15). Alteration in afferent neural signals is also manifested by a reduced capacity of IC neurons to transduce changes in preload. Third, autonomic efferent inputs to the ICNS are maintained post-MI (FIG. 15). Fourth, convergent IC LCNs, those receiving both afferent and efferent inputs, have enhanced transduction capacity following MI (FIG. 15). Fifth, functional network connectivity within the ICNS is reduced post-MI. Finally, MI reduces the response and alters the characteristics of IC neurons to ventricular pacing.

The only difference between control and MI animals was the presence of an infarct scar. Therefore, the structural and functional changes noted herein are likely to be attributable to the MI. The functional remodeling of the ICNS was studied, on average, six weeks after the creation of the MI. This represents a stable phase for autonomic adaptations and is beyond the acute phase remodeling, characterized by myocyte death and neural degeneration. Based on hemodynamic indices such as LV end-diastolic pressure and contractility, the animals were in a chronic compensated state and had not transitioned into overt heart failure. Further, neural activity was recorded from the VIV GP because it is primarily involved in control of ventricular function and its neural somata are located upstream from the infarct zone. Thus, the neural remodeling observed is not attributed to direct ischemic injury to the neurons.

MI induced morphological and phenotypic changes of neurons within the VIV GP, manifested by an enlargement of neurons and a decrease in cholinergic phenotype. Interestingly, neural enlargement was only observed in the VIV GP, DIV GP, and RMA GP, which preferentially exert influence over the ventricles, and not in the RA GP, which preferentially exerts influence over the atria. There was also increased expression of VIP in all of the ganglionated plexi following MI. VIP is an important modulator of cardiac function and has a potential role in afferent signaling. These structural changes are similar to those reported in intrathoracic extracardiac ganglia in both humans and animals models of ischemic cardiomyopathy. It has been speculated that this neural remodeling arises from release of neurotropic factors, as well as alterations in afferent and efferent neural signaling. Provided herein is direct functional evidence of MI-induced changes in ascending and descending inputs to the ICNS.

Neural Sensory Inputs from Infarct Border Zones

While structural/morphological remodeling of efferent neural signals post-MI has been extensively studied, little attention has been given to afferent neural signals originating from the injured and adjacent non-injured myocardial tissue. Anatomical and functional studies have identified unipolar neurons in IC ganglia with sensory neurites located in atrial and ventricular tissues. These afferent neurons transduce the local mechanical and chemical milieu of the heart. Afferent neurons contained within each GP have spatially divergent receptive fields, allowing for transduction of sensory information from widespread cardiac regions. Shown here is that VIV GP neurons, located adjacent the origin of the left anterior descending coronary artery (LAD) from the left main coronary artery, transduces sensory input arising from diverse cardiac regions overlying the right and left ventricles.

Following MI, myocardial necrosis occurs in the infarct zone secondary to ischemia. Further, a lack of energy substrates and a buildup of molecules such as reactive oxygen species trigger a cascade of intracellular signaling processes that result in remodeling of myocytes in the infarct border zone. Concurrent with myocyte remodeling, there are adaptive and maladaptive changes that occur at multiple levels of the cardiac neuraxis including the ICNS. In the acute state, there is excessive and aberrant activation of IC neurons transducing afferent signals from the injured myocardial tissue. Data presented herein demonstrates that in the chronic state, afferent signals from the infarct zone to the ICNS are reduced but not completely abolished, while those from border and remote zones are preserved. This heterogeneity in afferent neural signals gives rise to boundary conditions and a ‘neural sensory border zone’ analogous to the myocardial border zone induced by scar formation. The importance of sensory boundary conditions has already been shown in other neural circuits, such as the visual system where there is an enhanced ability for retinal ganglion cells to detect non-uniform light fields. It is believed that the MI-induced asymmetry in afferent inputs to the ICNS may underlie reflex activation of the ANS, including sympatho-excitation. In this regard, application of resiniferatoxin, a potent agonist of transient receptor potential vanilloid 1, post-MI decreases cardiac afferent nociceptive signaling, reduces sympatho-excitation, and is associated with preserved cardiac function. These data point to the fundamental importance of afferent neural signals in progression of cardiac disease and their role as a therapeutic target to manage the disease process.

Convergent Local Circuit Neurons: Information Processing within the Intrinsic Cardiac Nervous System

In this example, it is shown that a subpopulation of IC neurons, termed convergent LCNs, receive both afferent and efferent inputs. Convergent LCNs, together with afferent and efferent neurons, form the basic constituents of the IC neural circuitry. Within this circuit, convergent LCNs integrate and process information, and the presence of a large subpopulation of these neurons even in the MI state demonstrate that the capacity for local information processing is maintained. While there was no difference in the overall functional classification of neurons post-MI (afferent, efferent and convergent), the integrated network response to cardiovascular stimuli adapts/remodels. This was evident in the neural response to reduced preload and to regional pacing. Although most neurons that responded to pacing were also found to transduce dynamic cardiovascular changes, there was a unique subset that solely responded to pacing. The altered ICNS response to pacing at not only the infarct but also remote zones of the heart indicates that pacing may place additional stress on the ICNS on top of that imposed by the MI. Ventricular pacing in the presence of an infarct scar has been shown to have detrimental effects on cardiac function, including hypertrophy and dyssynchrony of the ventricles. Ultimately, this can speed up the progression to heart failure and increase mortality. The proposed mechanism underlying these adverse events relates to activation of the neuroendocrine system. Thus, it is believed that modulation of ICNS activity, in conjunction with MI-induced remodeling of afferent neural signals, could be a fundamental link between pacing and neuroendocrine system activation.

### Efferent Neural Control

The ICNS was classically viewed as a simple relay station for parasympathetic preganglionic efferent projections to the heart. Contrary to this view and in support of data obtained in the canine animal model, it is shown that a large percentage of porcine IC neurons received inputs from sympathetic or parasympathetic preganglionic neurons, as well as complex cardiovascular afferent inputs. The fact that a subset of IC neurons received a confluence of efferent inputs (inputs from both a stellate ganglia and vagus nerve) implies that a significant degree of sympathetic-parasympathetic interactions occur within the ICNS.

Remodeling of efferent neural signals following MI occurs at multiple levels of the cardiac neuraxis. At the organ level, sympathetic denervation of the infarcted myocardium and hyperinnervation of the border zones has been observed. Morphologic and neurochemical changes have also been noted in neurons contained within sympathetic ganglia such as the stellate. The increases in sympathetic influences are accompanied by a withdrawal in centrally-mediated parasympathetic influences. Despite these alterations in sympatho-vagal balance, the data presented herein demonstrates sympathetic and parasympathetic inputs to the ICNS remain intact post-MI. In fact, the percentage of IC neurons receiving convergent efferent inputs doubled following MI. The vast majority of these IC neurons, in turn, were local circuit in nature as evidenced by the fact that 90% of them were impacted by one or more afferent stimuli. This adaptation may be an attempt to maintain peripheral network stability in face of the destabilizing effects imposed by sympatho-vagal imbalance and the disparate afferent inputs arising from the infarct versus border and remote zones of the ventricle.

The present study provides direct evidence that the neural signature of the ICNS is altered in MI, and underlies the utility of neural recordings in helping elucidate the etiology of cardiac disease. The heterogeneity of afferent neural signals is likely fundamental to reflex activation of the ANS, thereby impacting the potential for arrhythmias and progression to heart failure. Modulation of afferent neural signals from the diseased myocardium to the ICNS, intrathoracic extracardiac ganglia, and higher centers of the cardiac neuraxis represents a novel therapeutic approach to mitigating ischemic heart disease. These findings also highlight that cardiac electroneurography could serve as an additional modality for study of cardiac physiology and pathophysiology.

The results found herein suggest chronic MI alters information processing in the ICNS. It was observed that a significant decrease in afferent signaling from the infarct zone to the ICNS. Although no change in evoked efferent signaling to the ICNS was observed, the integrated IC reflex response to IVC occlusion was significantly attenuated. These MI-induced alterations in information processing in the ICNS represent the ‘neural signature’ of altered cardiac afferent signaling, which initiates the pathophysiologic processes responsible for arrhythmias and heart failure.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of assessing ischemic heart disease in a subject, comprising:
measuring a plurality of electrical signals indicative of a neural signature from at least one intrinsic cardiac neuron;
comparing the measured signals to a reference neural signature of the intrinsic cardiac nervous system; and
determining if the difference between the measured signals and the neural signature exceeds a threshold value.

2. The method of claim 1, further comprising treating the subject with at least one therapeutic element when the threshold value is exceeded.

3. The method of claim 2, wherein the at least one therapeutic element is a drug or biological agent.

4. The method of claim 2, wherein the at least one therapeutic element is an electrical stimulus to a region of the subject's myocardial tissue or to one or more intrinsic cardiac neurons.

5. The method of claim 1, wherein the method comprises detecting the relative amount of afferent neurons in a population of intrinsic cardiac neurons.

6. The method of claim 1, wherein the reference neural signature is specific to the subject.

7. The method of claim 1, wherein the reference neural signature is based on a subject population having at least one common characteristic selected from the group consisting of gender, age, activity level, diet, congenital defect, genetic trait, and metabolic status.

8. The method of claim 1, wherein the measured neural signature includes at least one parameter selected from the group consisting of intrinsic neuron spontaneous firing rate, activity during cardiac cycle phases, temporal relationship between neurons, response to mechanosensitive input, response to stimulation of the sympathetic or parasympathetic nervous system, change in cardiac loading conditions, response to epicardial pacing, response to chemoreceptor stimulation, and response to noceptive input.

9. A method of treating a subject having diseased myocardium, comprising:
identifying diseased myocardial tissue in the subject's heart;
identifying at least one afferent intrinsic cardiac neuron signaling from the diseased myocardial tissue; and
modifying the signaling from the identified afferent intrinsic cardiac neuron.

10. The method of claim 9, wherein identifying at least one afferent intrinsic cardiac neuron signaling from the diseased myocardial tissue comprises comparing electrical signals measured from afferent neurons to a neural signature of the intrinsic cardiac nervous system.

11. The method of claim 9, wherein modifying the signaling from the identified afferent intrinsic cardiac neuron comprises applying an electrical stimulus to the afferent intrinsic cardiac neuron.

12. The method of claim 9, wherein the method is used in a closed loop system for monitoring and treating ischemic heart disease in the subject.
